(19) United States
(12) Patent Application Publication
(10) Pub. No.: US 2019/0242896 A1
(43) Pub. Date: Aug. 8, 2019

(54) SYSTEM AND METHOD FOR NONINVASIVELY ASSESSING BIOENGINEERED ORGANS

(71) Applicants: The University of North Carolina at Chapel Hill, Chapel Hill, NC (US); SonoVol, Inc., Research Triangle Park, NC (US)

(72) Inventors: Ryan Christopher Gessner, Durham, NC (US); Paul Alexander Dayton, Carrboro, NC (US); Max Stephan Harlacher, Durham, NC (US); James Owen Butler, Durham, NC (US); Tomasz Joseph Czernuszewicz, Chapel Hill, NC (US); Graeme Rainier O'Connell, Carrboro, NC (US)

(73) Assignees: The University of North Carolina at Chapel Hill, Chapel Hill (US); SonoVol, Inc., Research Triangle Park (US)

(21) Appl. No.: 16/327,726

(22) PCT Filed: Sep. 5, 2017
(86) PCT No.: PCT/US2017/050076

§ 371 Date: Feb. 22, 2019

Related U.S. Application Data
(60) Provisional application No. 62/383,068, filed on Sep. 2, 2016.

(51) Int. Cl. G01N 33/569; G01N 29/06; G01N 29/44; B33Y 40/00

(52) U.S. Cl. CPC G01N 33/56966; G01N 29/0654; B33Y 30/00; B33Y 40/00; G01N 2291/02475; G01N 29/44

(57) ABSTRACT

Provided are systems for analyzing cellular distribution in an engineered tissue sample, which optionally is a bioprinted organ or tissue sample. In some embodiments, the systems include an ultrasound imaging system and a processing unit configured with software that permits analysis of images acquired from the engineered tissue sample in order to output desired characteristics thereof. In some embodiments, the systems also include a bioreactor for engineering a tissue sample and a pump configured to regulate flow of fluids and reagents into and out of the bioreactor, wherein at least one surface of the bioreactor includes a window that is acoustically transparent to ultrasound waves. Also provided are systems for analyzing cell distribution in an engineered tissue sample and methods for analyzing distribution of cells in an engineered tissue sample present within a bioreactor.

### CROSS REFERENCE TO RELATED APPLICATION

This application claims the benefit of U.S. Provisional Application Ser. No. 62/383,068, filed Sep. 2, 2016, the disclosure of which is incorporated herein by reference in its entirety.

### GOVERNMENT INTEREST

This invention was made with United States government support under Grant No. IIP-1533978 awarded by the National Science Foundation of the United States. The United States government has certain rights in the invention.

### TECHNICAL FIELD

The presently disclosed subject matter relates generally to systems and methods for noninvasively evaluating engineered tissues and organs. In some embodiments, the presently disclosed subject matter relates to systems and methods for determining whether cells have localized and integrated into their intended location within a target tissue or organ.

### BACKGROUND

For many diseases, organ transplant remains the only viable option for saving a patient's life. Unfortunately, there is a massive shortage of available organs across the board, and many patients die before they get off the waiting list. For example, liver transplantation represents a fundamental therapy for patients suffering end-stage liver failure. Unfortunately, this intervention is limited due to a critical shortage of suitable donor organs. In the United States, the United Network for Organ Sharing reports more than 14,500 candidates currently awaiting liver transplant, with only 7,000 transplants being performed in the last year. This discrepancy between high-quality organ supply and demand results in, not only, organ wait-lists ranging from 12 to 36 months, but thousands of deaths each year of patients who simply run out of time.

In the last decade, advances in the fields of regenerative medicine and tissue engineering have led to the rise of a potential solution: whole-organ bioengineering, in which decellularized donor scaffolds (human or animal) are repopulated with the recipient's own cells. Envisioned is the possibility that organs can be generated on-demand and custom tailored to a patient's physiology, not only eliminating the need for transplant wait-lists but also the requirement of lifetime immunosuppression. While extremely promising, the field of whole-organ bioengineering is still in its infancy. Early studies have demonstrated that one of the primary barriers preventing successful implantation of organ constructs is the development of sufficiently patent and endothelialized vasculature. Without proper endothelial cell coverage, the organ's pro-thrombotic collagen matrix is left exposed to the host's circulating blood, resulting in the potential for obstructive clotting events and complete organ shutdown. Likewise, without proper perfusion, portions of the organ can become hypoxic and newly seeded cells will experience ischemia and cell death. The challenges posed by these vascularization problems are exacerbated by a slow experimental feedback loop driven by a lack of non-destructive tools for evaluating experimental success.

Despite the ubiquity of many noninvasive imaging technologies for examining humans and animal models (e.g., MRI, CT, PET, ultrasound, etc.), these systems are not utilized in any widespread capacity in the tissue engineering pipeline. There remains a strong desire in the field for better monitoring tools, but several challenges have precluded their use to date.

First, there are transportation challenges. Bioreactors and 3D printers employed in tissue engineering are typically coupled to at least one perfusion pump for circulating media and cells through the construct. This hardware and fluid circuitry is cumbersome to transport to imaging core facilities.

Second, there are difficulties with sample sensitivity. Engineered tissues must be kept sterile and maintained at physiological temperatures within laboratory incubators. Removing them from these carefully controlled environments can result in contamination and harm to the cells within. Furthermore, motion-induced jarring of tissue constructs can compromise their integrity.

Third, there are spatial and physical considerations that must be taken into account. Engineered organs are cultured within bioreactors or 3D printers of widely varying sizes, most of which cannot be placed into existing imaging systems and/or are non MRI compatible.

Therefore, bioengineered tissue and organ imaging represents a unique challenge; namely, that imaging should ideally come to the tissue rather than the tissue going to the imaging device.

Currently there are no widely available commercial tools for noninvasively evaluating bioengineered tissues. Specifically, one of the challenges of creating engineered organs is knowing whether cells have “seeded” or assimilated into their intended location within the organ. Because there is no noninvasive way of evaluating this, engineers and biologists in both academia and industry are required to invasively determine whether the seeding was successful. Methods for investigating this include biopsies, histological sections, etc., which significantly increases costs associated with organ and tissue engineering and dramatically lengthens the experimental feedback loop as histological analyses can take weeks.

A noninvasive imaging technology will also need to provide methods for cell tracking and serial monitoring during reseeding or 3D printing. Without noninvasive tools, researchers rely on methods such as histological analyses to determine the success of a given cell-seeding protocol. Not only does this incur additional costs and substantial time delays for every data point, it also necessitates that the tissue be sacrificed. Another driver of costs is the cells themselves; a human-sized organ requires researchers to grow hundreds of millions of cells and then perfuse them for a predetermined period of time through a scaffold using hundreds to thousands of dollars' worth of sterile media, without any knowledge whatsoever about their seeding efficacy in real time. Similar costs are present in the case of 3D printing of tissues or organs. If there was an intermediate feedback mechanism to tell them how the experiment was proceeding, researchers could pivot to a new set of parameters without needlessly wasting weeks of their lab's time and thousands of dollars on a doomed study.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

In some embodiments, the presently disclosed subject matter provides systems for analyzing cell distribution in engineered tissue samples, optionally wherein the engineered tissue samples are present within a bioreactor. In some embodiments, the systems comprise (a) an imaging system comprising at least one ultrasound transducer for acquiring ultrasound images from an engineered tissue sample present in the bioreactor; and (b) a processing unit configured to analyze the ultrasound images acquired by the ultrasound transducer from the engineered tissue sample in order to output measured characteristics of the engineered tissue sample. In some embodiments, the ultrasound transducer is configured be located external to the bioreactor when acquiring the ultrasound images. In some embodiments, the ultrasound transducer is configured to obtain the ultrasound images through an acoustically transmissive window in the bioreactor. In some embodiments, the ultrasound transducer is configured to be located in the bioreactor when acquiring the ultrasound images. In some embodiments, the bioreactor comprises a three dimensional printer for generating the engineered tissue sample through a three dimensional printing process. In some embodiments, the ultrasound transducer is interchangably couplable to the three dimensional printer for acquiring the ultrasound images. In some embodiments, the ultrasound transducer is separate from the three dimensional printer for acquiring the ultrasound images. In some embodiments, the ultrasound transducer is configured to generate ultrasound energy to image an ultrasound contrast agent configured to bind to the engineered tissue sample. In some embodiments, the processing unit is configured to output an indication of an amount of the ultrasound contrast agent bound to the engineered tissue sample.

In some embodiments, the presently disclosed subject matter provides systems for analyzing cell distribution in engineered tissue samples. In some embodiments, the systems comprise (a) a bioreactor for generating an engineered tissue sample, wherein the bioreactor (i) comprises an interior region for holding the engineered tissue sample; (ii) comprises one or more input lines and one or more exit lines, both in fluid communication with the bioreactor for introducing a fluid into the interior region and removing the fluid from the interior region; and (iii) comprises a window that is transmissive to ultrasound waves; (b) a pump connected to at least one of the one or more input lines and/or to at least one of the one or more exit lines configured to regulate flow of the fluid into and out of the interior region; and (c) an imaging system comprising at least one ultrasound transducer for acquiring ultrasound images from the engineered tissue sample present in the bioreactor. In some embodiments, the presently disclosed systems further comprise a processing unit configured to analyze the ultrasound images acquired from the engineered tissue sample in order to output measured characteristics of the engineered tissue sample. In some embodiments, at least one of the one or more input lines comprises an inlet port configured to permit introduction of a reagent into the fluid under conditions such that the reagent perfuses the engineered tissue sample. In some embodiments, the reagent comprises a contrast agent. In some embodiments, the contrast agent comprises a ligand that specifically binds to a target molecule present in the engineered tissue sample. In some embodiments, the ligand comprises an antibody or an antigen-binding fragment thereof that specifically binds to the target molecule. In some embodiments, the target molecule is present in the engineered tissue sample and is accessible to the ligand in locations of the engineered tissue sample that are decellularized or non-cellularized. In some embodiments, the ligand binds to a collagen matrix present in a decellularized or non-cellularized region of the engineered tissue sample. In some embodiments, the target molecule is present in the engineered tissue sample and is accessible to the ligand in locations of the engineered tissue sample that are recellularized. In some embodiments, the target molecule is present in the engineered tissue sample only in locations of the engineered tissue sample that are recellularized. In some embodiments, the target molecule is a molecule expressed by an endothelial cell. In some embodiments, the molecule expressed by an endothelial cell is selected from the group consisting of CD31, P-selectin, E-selectin, VEGF-R2, and αᵥβ₃ integrin. In some embodiments, the flow of the fluid in the bioreactor is interruptible to stop perfusion of the engineered tissue sample. In some embodiments, the fluid carries an ultrasound contrast agent through the engineered tissue sample, and wherein at least one of the at least one exit lines is configured to selectively route output of fluid from the bioreactor to remove a portion of the contrast agent that does not bind with the engineered tissue sample from the bioreactor.

In some embodiments, the presently disclosed subject matter also provides systems wherein the engineered tissue sample comprises a liver scaffold, a lung scaffold, or a kidney scaffold. In some embodiments, the engineered tissue sample is decellularized. In some embodiments, the engineered tissue sample comprises a bioprinted organ or tissue.

In some embodiments of the presently disclosed systems, the ultrasound transducer is connected to the bioreactor via a docking mechanism that permits two-dimensional or three-dimensional movement of the ultrasound transducer relative to the engineered tissue sample.

In some embodiments of the presently disclosed systems, the ultrasound transducer is capable of receiving ultrasound signals of >5 MHz.

In some embodiments of the presently disclosed systems, the processing unit is configured to accept ultrasound image input and output percent cellularization of the engineered tissue sample.

In some embodiments, the presently disclosed subject matter provides methods for analyzing distribution of cells in engineered tissue samples, optionally wherein the engineered tissue samples are present within a bioreactor. In some embodiments, the methods comprise (a) introducing a contrast agent to perfusion input of the engineered tissue sample, wherein the contrast agent specifically binds to a target molecule expressed by endothelial cells present within the engineered tissue sample or to a target molecule present in a decellularized region of the engineered tissue sample; (b) permitting the contrast agent to contact the engineered tissue sample under conditions and for a time sufficient to allow binding of the contrast agent to the target molecule, if present; and (c) acquiring image data of the engineered tissue sample, wherein the image data allows for a determination of whether or not the contrast agent has bound to the engineered tissue sample in one or more regions of the engineered tissue sample.

In some embodiments, the presently disclosed methods further comprise processing the acquired image data using a processing unit capable of transforming the acquired image data into output indicative of one or more regions of the engineered tissue sample where endothelial cells are or are not present. In some embodiments, the acquired image data is outputted as spatial density of endothelial cells based on the image of stationary contrast agents, optionally in comparison to reference image data. In some embodiments, the acquired image data is outputted as spatial density of decellularized regions of the engineered tissue sample, thereby providing a map of a network of decellularized vasculature of the engineered tissue sample. In some embodiments, the engineered tissue sample comprises a bioprinted organ or tissue sample.

The presently disclosed subject matter also provides in some embodiments methods for analyzing bioprinted organ and/or tissue samples. In some embodiments, the presently disclosed methods comprise (a) introducing a contrast agent to perfusion input of the bioprinted organ or tissue sample, wherein the contrast agent specifically binds to a target molecule expressed by cells present within the bioprinted organ or tissue sample, (b) permitting the contrast agent to contact the bioprinted organ or tissue sample under conditions and for a time sufficient to allow binding of the contrast agent to the target molecule, if present; and (c) acquiring image data of the bioprinted organ or tissue sample, wherein the image data allows for a determination of whether or not the contrast agent has bound to the bioprinted organ or tissue sample in one or more regions of the bioprinted organ or tissue sample. In some embodiments, the presently disclosed methods further comprise processing the acquired image data using a central processing unit programmed with software capable of transforming the acquired image data into output of one or more regions of the bioprinted organ or tissue sample where cells are or are not present. In some embodiments, the acquired image data is outputted as spatial density of cells based on the image of stationary contrast agents, optionally in comparison to reference image data. In some embodiments, the acquired image data is outputted as spatial density of non-cellularized and/or incompletely cellularized regions of the bioprinted organ or tissue sample, thereby providing a map of a network of non-cellularized and/or incompletely cellularized regions of the bioprinted organ or tissue sample. In some embodiments, acquiring the image data includes using an ultrasound transducer located external to a bioreactor in which the bioprinted organ or tissue sample is located. In some embodiments, acquiring the image date includes using an ultrasound transducer located inside of a bioreactor in which the bioprinted organ or tissue sample is located.

An object of the presently disclosed subject matter having been stated hereinabove, and which is achieved in whole or in part by the presently disclosed subject matter, other objects will become evident as the description proceeds when taken in connection with the accompanying Figures as best described herein below.

### BRIEF DESCRIPTION OF THE FIGURES

FIG. 1 is a schematic diagram of an exemplary embodiment of the presently disclosed subject matter. Depicted is a tissue enclosed within a sealed bioreactor. Perfusion inputs and outputs in fluid communication with the tissue are shown, with fluid flow provided by a pump system and flow directions indicated with arrows. A probe (e.g., an ultrasound probe) is coupled to the bioreactor in such a way that the probe can image the tissue present in the bioreactor. A contrast infusion apparatus is in communication with the pump system to introduce contrast into the fluid flow, as desired. Also depicted is hardware (e.g., a processing unit, a computer, etc.) that can be programmed to regulate the actions of the pump system and/or the contrast infusion apparatus, as well as an imaging system (also optionally controlled by the hardware) to accepting input from the probe and providing output to the hardware.

FIG. 2 is a schematic diagram of an exemplary embodiment of the presently disclosed subject matter. FIG. 2 depicts the ex vivo tissue scaffold present within a prototype bioreactor. The prototype bioreactor includes an acoustic window between the detection device (e.g., an ultrasound transducer) to capture images of the scaffold. The exemplary device can also include an input pump and an output pump in fluid communication with the bioreactor to provide fluid flow (e.g., media, contrast reagents, etc.) across the scaffold. Also depicted is a 3D motion stage that permits the detection device to be moved into various different positions in space relative to the scaffold. As shown in FIG. 2, the organ scaffold can be positioned within a bioreactor over the 3D imaging system's robotics. Infusion pumps perfuse and drain the organ scaffold while an acoustically transparent window couples the sound to the tissue.

FIG. 3 is a depiction of an exemplary apparatus of the presently disclosed subject matter. The left and right panels together are views showing a tissue scaffold enclosed within a sealed bioreactor. A perfusion input to the tissue and a perfusion output from the tissue is in fluid communication therewith. An acoustically transmissive membrane is located between the tissue scaffold and an ultrasound transducer such that the ultrasound transducer can image the tissue scaffold. Also depicted is a docking mechanism into which the ultrasound transducer can be fitted in order to secure the ultrasound transducer in position relative to the tissue scaffold.

FIG. 4 is a schematic diagram of an exemplary embodiment of the presently disclosed subject matter. FIG. 4 depicts a plurality of bioreactors present within an incubator to which a modular 3D imaging unit and an exemplary embodiment of the presently disclosed subject matter (Mobile OrganVis Device) can be brought into contact in order to image tissue samples present within the bioreactors. This design would allow contrast infusion pumps and robotic control systems to be housed within the interior of the incubator, with an external modular 3D imaging unit capable of docking to the bioreactor(s) located within the incubator at various heights relative to the floor. Thus, an advantage of the presently disclosed subject matter is that in some embodiments the imaging unit and the analysis unit (e.g., the Mobile OrganVis Device) can be portable such that they can be moved into proximity to the bioreactors (optionally present in an incubator) so that the bioreactors per se would experience little or no movement, thereby resulting in minimal disturbance of the tissue samples present in the bioreactors during the imaging process.

FIG. 5 depicts an exemplary microbubble contrast agent (MCA) of the presently disclosed subject matter. In some embodiments, an MCA comprises a lipid shell surrounding a gas core. The lipid shell is functionalized to comprise a targeting moiety that binds specifically to target cells under the conditions encountered in the bioreactor. In some embodiments, the target cell is an endothelial cell and the targeting moiety is a molecule (e.g., an antibody or a fragment or derivative thereof that comprises a paratope) that binds to a ligand expressed by the endothelial cell (e.g., CD31, P-selectin, E-selectin, VEGF-R2, or αᵥβ₃ integrin).

FIG. 6 is an exemplary schematic depiction of the steps involved in molecular imaging with ultrasound of the presently disclosed subject matter. As depicted in FIG. 6, a bioreactor comprising a tissue scaffold to be imaged is docked to an imaging system of the presently disclosed subject matter. A b-mode image of the tissue scaffold in three dimensions is acquired in the absence of contrast agents. One or more contrast agents (e.g., one or more MCAs) are infused into the bioreactor under conditions sufficient to allow the one or more contrast agents to interact with and/or bind to their targets (e.g., endothelial cells present within the vessel trees of the tissue scaffold). Contrast-enhanced images of the vessel trees are then acquired, and the bioreactor is returned to the incubator. The acquired contrast-enhanced images of the vessel trees are processed and desired output variables (e.g., the percentage of the vessel tree seeded with endothelial cells) are derived. Using the systems, compositions, and methods of the presently disclosed subject matter, this entire process can be completed in less than five minutes with no harm to the tissue sample. Also importantly, these manipulations can be performed without negatively impacting the sterile environment of the bioreactor.

FIGS. 7A and 7B are depictions of blood vessels lined with endothelial cells (orange ovals) expressing an endothelial marker (block protrusions from the endothelial cells) and targeting by MCAs of the presently disclosed subject matter (black circles coated with gray protrusions). In FIG. 7A, freely-flowing targeted microbubbles (i.e., MCAs) are shown in the lumen of the vessel, unbound to any targets. FIG. 7A depicts the state after injection of the contrast agent containing the MCAs but before the MCAs can bind to their targets. FIG. 7B depicts targeting of the MCAs to the endothelial marker target expressed by the endothelial cells. (e.g., about 5 minutes after introduction of the contrast agent into the bioreactor in which the blood vessels are present). The lack of free MCAs present in the lumen of the vessel also indicates a state where sufficient time has elapsed to allow unbound MCAs to be removed from the media circulating through the vessel.

FIG. 8 depicts an exemplary technique for molecular imaging with ultrasound of the presently disclosed subject matter that delineates stationary from moving contrast agents. In the right panel, three forms of microbubble contrast agents (MCAs) are depicted. MCA1 represents an MCA that is moving and not bound to a cell. MCA2 represents an MCA that is moving and bound to a cell. MCA2 thus represents an MCA that is bound to a cell that is not seeded. MCA3 represents an MCA that is bound to a cell and not moving. MCA3 thus represents an MCA that is bound to a cell that is seeded. If the stationary signal data entirely align to the 3D vessel data, the user knows that the entire vessel network has endothelial coverage.

FIGS. 9A-9C depict a representation of the output of the presently disclosed imaging and analysis methods for assaying seeding of an engineered liver scaffold. FIG. 9A illustrates a microvascular network within an ex vivo rat liver scaffold. FIG. 9B is a simulated image displaying what a CD31 endothelial cell molecular targeted image would look like for the scaffold seen in FIG. 9A if it were not fully seeded with endothelial cells. FIG. 9C is a simulated composite overlay of the images presented in FIGS. 9A and 9B. The composite can be used to determine a “percent endothelialization”, which in some embodiments could be calculated as [(the area deemed to be positive in FIG. 9B divided by the area deemed to be positive in FIG. 9A)×100%]. If the area deemed to be positive in FIG. 9B appeared identical to the area deemed to be positive in FIG. 9A, then the scaffold would be deemed to be “completely endothelialized” or “completely seeded”.

FIGS. 10A-10D depict an overview of a study employing the presently disclosed subject matter both during decellularization and recellularization of an liver scaffold. FIG. 10A depicts an exemplary study for mapping changes in endothelial cell coverage during decellularization. An explanted rat liver is flushed with basal medium, delipidized, and extracted with high salt to decellularize the liver scaffold. At various time points (TP) during this process, the liver explant can be imaged with the systems and methods of the presently disclosed subject matter to monitor the decellularization process. FIGS. 10B and 10C illustrate how endothelialization (“seeding”) can be quantified via ultrasound and histology, respectively. The vessel images in FIG. 10B include endothelial mapping simulated on top. FIG. 10D is a graph of simulated data illustrating a correlation between the non-invasive ultrasound of the presently disclosed subject matter and the gold standard histological analysis of greater than 0.8 for the six (6) time points shown in FIG. 10A.

FIGS. 11A and 11B depict exemplary embodiments of a bioreactor chamber (FIG. 11A) and a secondary bypass circuit (FIG. 11B) that can be employed for removing contrast agents from the bioreactor.

FIGS. 12A and 12B depict exemplary embodiments of an imaging system of the presently disclosed subject matter that can be employed to image an engineered organ or tissue, in some embodiments a 3D printed organ or tissue. FIG. 12A depicts an embodiment of the presently disclosed subject matter in which the imaging system is fixed to a robotically controlled printing head of a 3D printing device adapted to produce an engineered organ or tissue. In some embodiments when used in this configuration, the robotic printing arm employs the ultrasound scanner to evaluate printed tissues by coupling the transducer to the surface of the tissue. FIG. 12B depicts an embodiment of the presently disclosed subject matter in which the imaging system is not fixed to the robotically controlled printing head of the 3D printing device adapted to produce the engineered organ or tissue, but rather is placed at a location in the vicinity of the engineered organ or tissue in order to image the engineered organ or tissue. In some embodiments where the imaging system is not fixed to the robotically controlled printing head of the 3D printing device, the imaging system can be placed in any position whereby imaging of an appropriate region of the engineered organ or tissue is performed. Alternatively or in addition, the imaging system can be adapted to rotate around the engineered organ or tissue to image several different regions of the same and/or from several different spatial locations.

FIG. 13 depicts isolation of a pulmonary vessel segment from a pig lung and fitting the same into a holder for manipulation and imaging using an imaging system of the presently disclosed subject matter. In the left panel, a pig lung sample is depicted. A pulmonary vessel segment of about 1 cm in length has been isolated from the lung sample and inserted into a holder. The holder includes a tube with an open end into which the pulmonary vessel segment is inserted. The holder also includes a barbed fitting at one end to which silicon tubing is attached (right panel).

FIG. 14 depicts the holder containing the pulmonary vessel segment shown in FIG. 13 placed in a bath for imaging using the imaging system of the presently disclosed subject matter. The ultrasound probe is placed adjacent to the holder containing the pulmonary vessel segment in a position whereby the pulmonary vessel segment can be imaged. The barbed fitting fixes the orientation of the pulmonary vessel segment in the holder so that fluids introduced into the holder flow through the lumen of the pulmonary vessel segment. These fluids can be introduced by attaching a syringe apparatus to the holder via the silicon tubing. In addition, the syringe apparatus can include one or more ports for introducing additional fluids (e.g., targeted contrast agents” into the fluid flow provided by the syringe apparatus. In this arrangement, the external surface and the lumen of pulmonary vessel segment can be washed (e.g., with phosphate-buffered saline, PBS) before and/or after introduction of a contrast agent. The contrast agent can be introduced into the lumen of the pulmonary vessel segment while the pulmonary vessel segment is in a fixed position in relation to the ultrasound probe, allowing the contrast agent to bind to its target(s).

FIG. 15 depicts the arrangement of the ultrasound probe and the ultrasound beam produced thereby in relation to the tissue sample. As shown in the left panel, the syringe provides fluid flow in the direction of the dashed arrow, which traverses the lumen of the pulmonary vessel segment. In some embodiments, the ultrasound beam is oriented in a direction such that it images the pulmonary vessel segment perpendicular to its axis (see FIG. 15, left panel), resulting in a vessel cross section image if the contrast agent binds to a target located on the interior surface of the lumen of the pulmonary vessel segment (see FIG. 15, right panel).

FIGS. 16A and 16B show representative examples of tissue imaging using an imaging system of the presently disclosed subject matter. FIG. 16A depicts b-mode imaging of the tissue in the absence of contrast agent. B-mode imaging can be useful for delineating the borders of a tissue sample. FIG. 16B is an image after introduction of a microbubble contrast agent (MCA). The introduction of a contrast agent into the tissue allows for imaging of the contrast agent within the lumen of the tissue as well as bound to targets (e.g., endothelial cells) present therein. For microbubbles to be delineated from tissue, in some embodiments a contrast-specific mode (e.g., Cadence Pulse Sequence, CPS) can be employed and is depicted in FIG. 16B.

FIGS. 17A-17D depict four (4) representative images of a vessel imaged with the imaging system of the presently disclosed subject matter. FIG. 17A is a b-mode image that shows a cross section of the vessel. FIG. 17B is an image of the vessel when fully perfused with targeted MCAs of the presently disclosed subject matter. FIG. 17C is an image of the vessel after a saline flush to remove unbound contrast agent. As can be seen by the absence of signal in the center of the image, the interior of the vessel has been cleared of unbound microbubbles, whereas targeted microbubbles remain bound to endothelial cells. FIG. 17D is a control image showing what the vessel looked like after the targeted microbubbles were destroyed, thereby illustrating that there was some ambient signal from bubbles in the media but that it was substantially less than that produced by the targeted microbubbles when bound to their cellular targets on the wall of the vessel.

FIG. 18 is a series of images similar to those depicted in FIG. 17 taken at four (4) different locations along the length of the vessel. From top to bottom, the groups of images are b-mode images of the tissue, images that show free and bound MCAs, images that show bound MCAs after removal of unbound MCAs, and controls after the targeted microbubbles present in the MCAs were destroyed. This Figure shows that the images are consistent along the vessel's length, demonstrating that flow of the MCA along the vessel efficiently labels the vessel along its length for imaging.

FIG. 19 is a juxtaposition of rows 3 (top panel) and 1 (bottom panel) of FIG. 18, highlighting the efficiency at which endothelial cells along pulmonary vessel walls can be imaged using the 3D non-invasive visualization systems of the presently disclosed subject matter.

FIGS. 20A-20C present another example of a vessel imaged with the imaging system of the presently disclosed subject matter. FIG. 20A depicts a three-dimensional image of a vessel that can be produced from the data acquired by an imaging system of the presently disclosed subject matter using a microbubble contrast agent (MCA) that binds to a target on the inner luminal surface of a vessel. FIG. 20B is a cross sectional representation of the same vessel depicted in FIG. 20A. The dashed line is indicative of the inner luminal surface. FIG. 20C is a graph of distance along a vessel axis in millimeters from an arbitrary start point versus the percent of the vessel wall that to which targeted microbubbles have bound. As can be seen from FIG. 20C, a fairly consistent level of cellular distribution was observed over the measured length of the vessel.

FIG. 21 depicts another example of an imaging system of the presently disclosed subject matter as employed to image an explanted porcine kidney sample. The kidney sample (organ) is immobilized in a holder, with the holder connected to a robotic stage that can move the kidney sample relative to the ultrasound transducer in two dimensions (axes of robotic stages).

FIG. 22 are images of the kidney sample of FIG. 21 using an exemplary imaging system of the presently disclosed subject matter. The gray areas correspond to kidney tissue. The black arrows indicate areas of accumulation of contrast along vessel walls in the kidney sample (the corresponding regions appear yellow in the corresponding color images).

### DETAILED DESCRIPTION

All references listed below, as well as all references cited in the instant disclosure, including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.

The present subject matter will now be described more fully hereinafter with reference to the accompanying Figures, in which representative embodiments of the presently disclosed subject matter are shown. The presently disclosed subject matter can, however, be embodied in different forms and should not be construed as limited to the embodiments set forth herein. Rather, these embodiments are provided so that this disclosure will be thorough and complete, and will fully convey the scope of the presently disclosed subject matter to those skilled in the art.

Reference will now be made in detail to the description of the present subject matter, one or more examples of which are shown in the Figures. Each example is provided to explain the subject matter and not as a limitation. In fact, features illustrated or described as part of one embodiment can be used in another embodiment to yield still a further embodiment. It is intended that the present subject matter cover such modifications and variations. Wherever possible, the same reference numbers will be used throughout the Figures to refer to the same or like parts. The scaling of the Figures does not represent precise dimensions of the various elements illustrated therein.

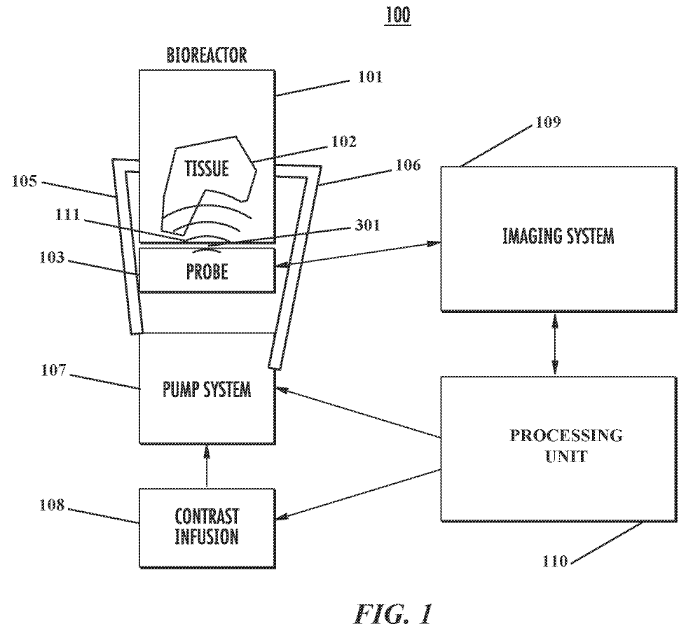

Referring now to the Figures, again wherein like reference numerals refer to like parts throughout when possible, exemplary embodiments of the presently disclosed subject matter are referred to generally at 100, 200, 300, 400, and 1100. Referring in particular to FIGS. 1-4 and 11, exemplary systems 100, 200, 300, 400, and 1100 can include multiple components. In various embodiments, exemplary systems 100, 200, 300, 400, and 1100 can include bioreactor 101 in which tissue (e.g., engineered tissue scaffold) 102 is present. In order to image engineered tissue scaffold 102 within bioreactor 101, probe (e.g., an ultrasound transducer) 103 can be fitted to bioreactor 101 via docking mechanism (e.g., coupling) 104. Probe 103 transmits ultrasound beams via ultrasound beam path 111 into a tissue 102 in order to image the same. Bioreactor 101 can also include one or more input lines 105 and one or more exit lines 106 in fluid communication therewith for introducing a fluid (e.g., a growth medium) into bioreactor 101 and for removing the fluid from bioreactor 101, respectively. One or more input lines 105 and one or more exit lines 106 can be controlled by pump system 107, which in some embodiments includes contrast infusion mechanism 108. Contrast infusion mechanism 108 can also be in fluid communication with one or more of the input lines 105, for example as an inlet port configured to permit introduction of a reagent into the fluid under conditions such that the reagent perfuses the engineered tissue sample present in bioreactor 101. Exemplary system 100 also includes in some embodiments imaging system 109, which is in communication with probe (e.g., ultrasound transducer) 103 to receive imaging data from probe (e.g., ultrasound transducer) 103. In some embodiments, one or more components of exemplary system 100 are controlled by processing unit 110. Components that can be controlled by processing unit 110 include pump system 107 (which in some embodiments can be processing unit or computer controlled with respect to turning fluid flow on or off as well as regulating the speed of fluid flow when pump system 107 is on), contrast infusion mechanism 108, and/or imaging system 109, which in some embodiments comprises an image analysis component and/or an image processing component.

With particular reference to FIG. 1, exemplary system 100 can include bioreactor 101, engineered tissue scaffold 102 within bioreactor 101, probe (e.g., ultrasound transducer) 103, and pump system 107 with input pump line 105 and exit pump line 106. In some embodiments, all or substantially all of bioreactor 101 is made of an acoustically transmissive material, which allows ultrasound beam path 111 into a tissue 102 in order to image the same. In some embodiments, it can also be desirable to image engineered tissue scaffold 102 from several directions by mounting probe (e.g., ultrasound transducer) 103 on a 3D motion stage. Bioreactor 101 can also comprise external coupling reservoir 301, which in some embodiments functions to maintain acoustic contact between the bioreactor and the imaging transducer, in some embodiments while the imaging transducer is being moved to different positions with respect to the bioreactor by the 3D motion stage. In some embodiments, pump system 107 is in fluid communication with contrast infusion reservoir 108 such that contrast agents can be delivered to bioreactor 101 and engineered tissue scaffold 102. In some embodiments, probe 103 is in communication with imaging system 109 such that images received by probe 103 can be delivered to and, in some embodiments, manipulated by imaging system 109. Imaging system 109 is also in communication with and in some embodiments under control of processing unit 110. In some embodiments, processing unit 110 is also in communication with and optionally controls contrast infusion reservoir 108 and/or pump system 107.

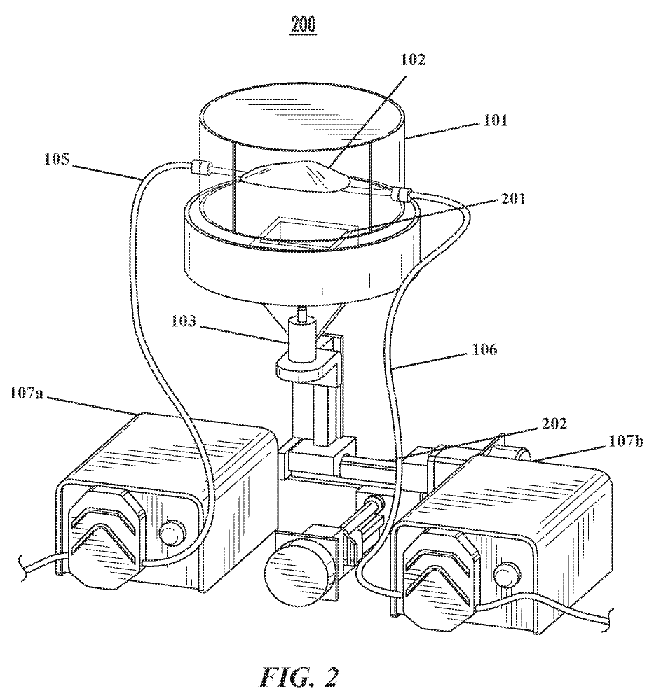

With particular reference to FIG. 2, exemplary system 200 can include bioreactor 101, engineered tissue scaffold 102 within bioreactor 101, probe (e.g., ultrasound transducer) 103, input pump 107a, and output pump 107b. In some embodiments, probe (e.g., ultrasound transducer) 103 is placed against acoustic window 201 present on one surface of bioreactor 101 such that engineered tissue scaffold 102 present in bioreactor 101 can be imaged. In some embodiments, acoustic window 201 is present over some or all of the surface of bioreactor 101 such that probe (e.g., ultrasound transducer) 103 can be placed against a plurality of surfaces in three-dimensional space of bioreactor 101 to image engineered tissue scaffold 102 from a plurality of different directions. In some embodiments, all or substantially all of bioreactor 101 is made of an acoustically transmissive material such that acoustic window 201 comprises all or substantially all of bioreactor 101. In some embodiments, it can also be desirable to image engineered tissue scaffold 102 from several directions by mounting probe (e.g., ultrasound transducer) 103 on 3D motion stage 202.

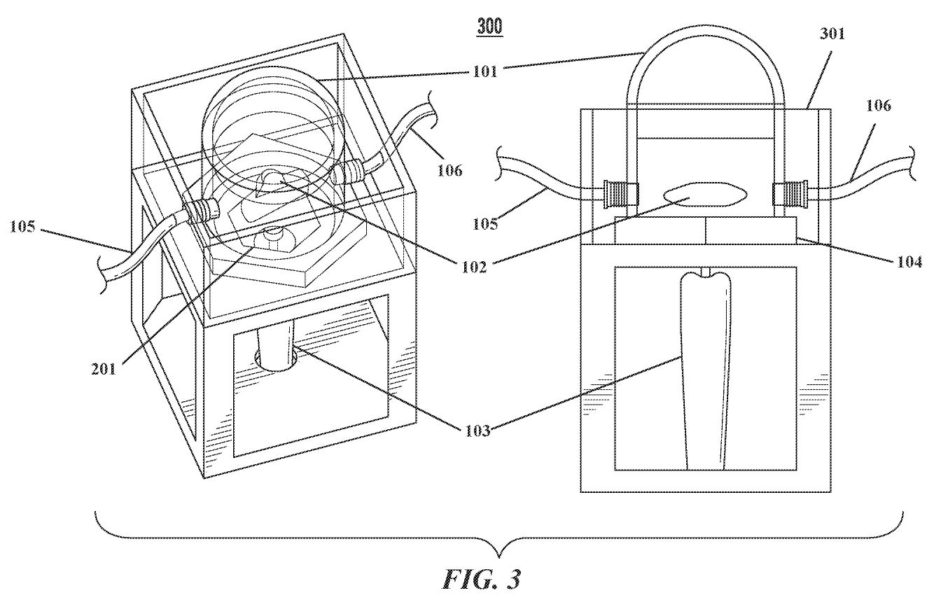

With particular reference to FIG. 3, exemplary system 300 can include bioreactor 101 with engineered tissue scaffold 102 present therein, probe (e.g., ultrasound transducer) 103 positioned to image engineered tissue scaffold 102 via docking mechanism 104. Acoustic window 201 present between probe (e.g., ultrasound transducer) 103 and engineered tissue scaffold 102 can be an acoustically transmissive membrane. Input line 105 and exit line 106 can provide perfusion input to and perfusion output from engineered tissue scaffold 102, respectively. Bioreactor 101 can also comprise external coupling reservoir 301, which in some embodiments functions to maintain acoustic contact between the bioreactor and the imaging transducer, in some embodiments while the imaging transducer is being moved to different positions with respect to the bioreactor.

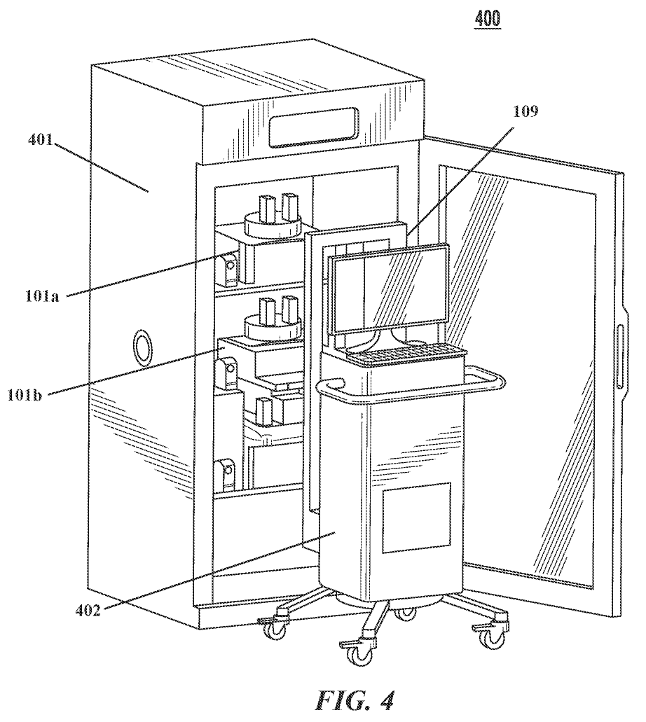

With particular reference to FIG. 4, exemplary system 400 can include incubator 401 in which bioreactors 101a and 101b are present. Imaging system 109 can be a modular 3D imaging unit as shown, with mobile OrganVis device 402 comprising modular 3D imaging unit 109 such that mobile OrganVis device 402 can be moved to incubator 401 so that bioreactors 101a and 101b need not be removed therefrom when imaged by modular 3D imaging unit 109.

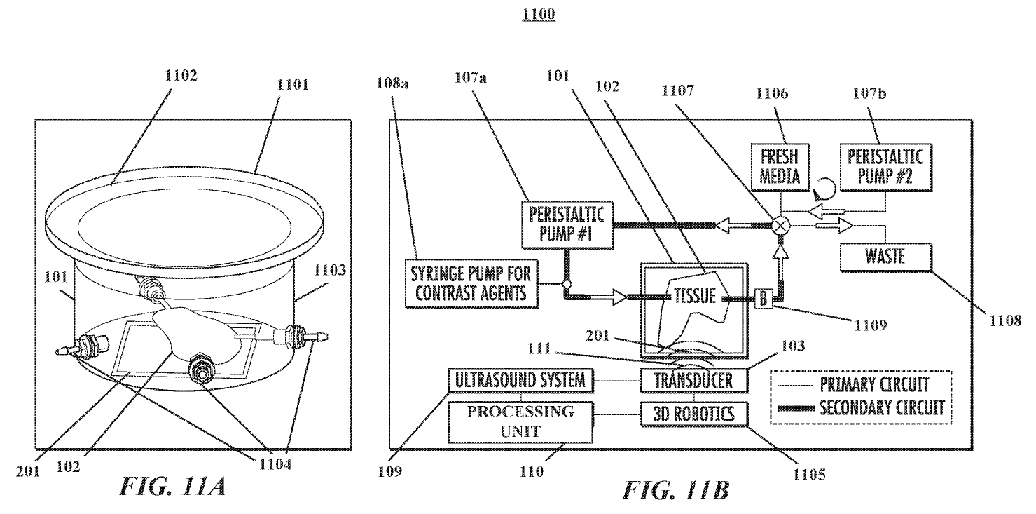

With particular reference to FIGS. 11A and 11B, exemplary system 1100 can include bioreactor 101 that includes top plate 1101, silicon sealing gasket 1102, and polycarbonate housing 1103. Exemplary system 1100 can also include one or more coupling ports 1104 that permit fluid communication from outside of bioreactor 101 to tissue 102, which can be adjacent to acoustic window 201. In some embodiments, ultrasound system 109 includes probe (e.g., ultrasound transducer) 103, which can also be attached to 3D robotics stage 1105 so that probe (e.g., ultrasound transducer) 103 can be moved in three dimensions to image different aspects of tissue 102. In some embodiments, the movement of probe (e.g., ultrasound transducer) 103 via 3D robotics stage 1105 is controlled by processing unit 110. Probe (e.g., ultrasound transducer) 103 can introduce ultrasound beam path Ill into tissue 102 through acoustic window 201 in order to image the same. Bioreactor 101 can be in fluid communication with input pumps 107a and 107b (e.g., peristaltic pumps #1 and #2) whereby fluids from fresh media reservoir 1106 and contrast agents from syringe pump for contrast agents 108b can be introduced into bioreactor 101. Between fresh media reservoir 1106 and bioreactor 101 can be bypass valve 1107, which can direct fluids exiting bioreactor 101 to waste receptacle 1108. In some embodiments, microbubble detector 1109 is placed between bioreactor 101 and bypass valve 1107 in order to facilitate the detection of contrast agents exiting bioreactor 101. Bypass valve 1107 can also regulate reinfusion of fluid into bioreactor 101 via a secondary circuit (thicker black line with associated arrows) or if desired can direct fluid from bioreactor 101 to waste receptacle 1108 via a primary circuit (thinner black line with associated arrows).

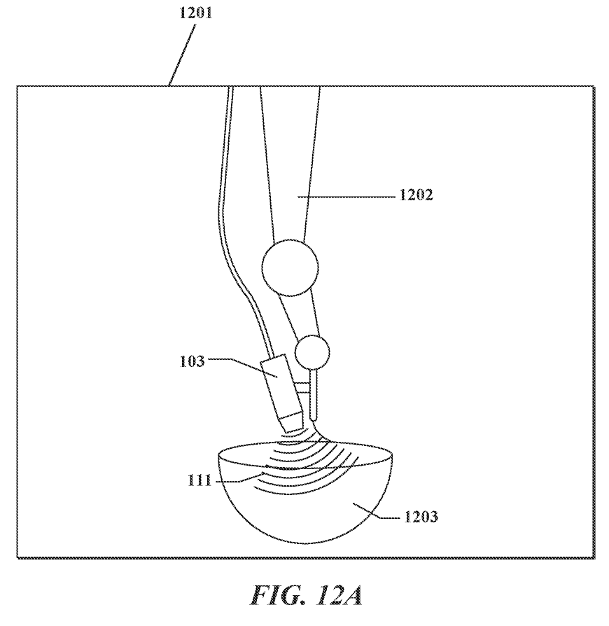

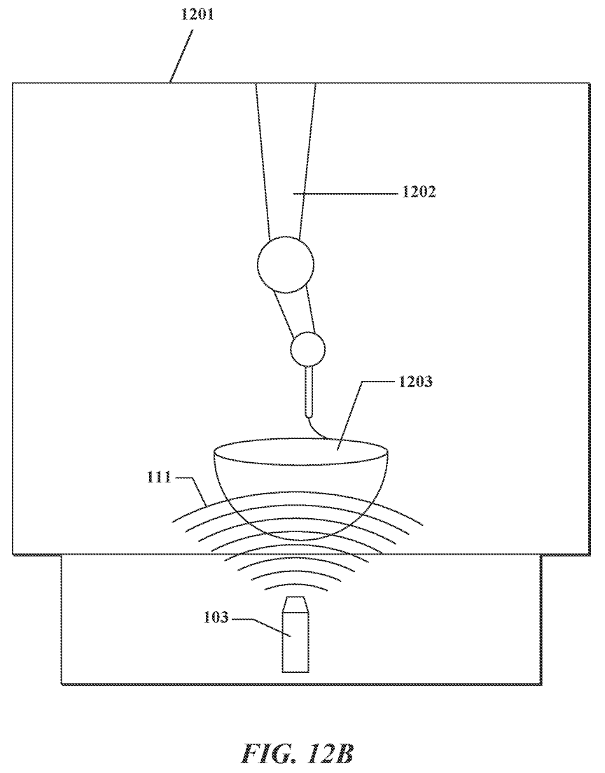

With particular reference to FIGS. 12A and 12B, the exemplary systems of the presently disclosed subject matter can also be employed for imaging 3D printed (i.e., bioprinted) tissues and organs. By way of example and not limitation, bioprinted tissue 1203 can be bioprinted using biomaterial printing head and robotic control with robotic control 1202 within sealed printing chamber 1201. In some embodiments, probe (e.g., ultrasound transducer) 103 is fixedly attached to biomaterial printing head and robotic control with robotic control 1202 in order to track the progress of the bioprinting process by introducing ultrasound beam path 111 into bioprinted tissue 1203. Alternatively or in addition, probe (e.g., ultrasound transducer) 103 can be placed adjacent to bioprinted tissue 1203 such that it can be moved independently from biomaterial printing head and robotic control with robotic control 1202.

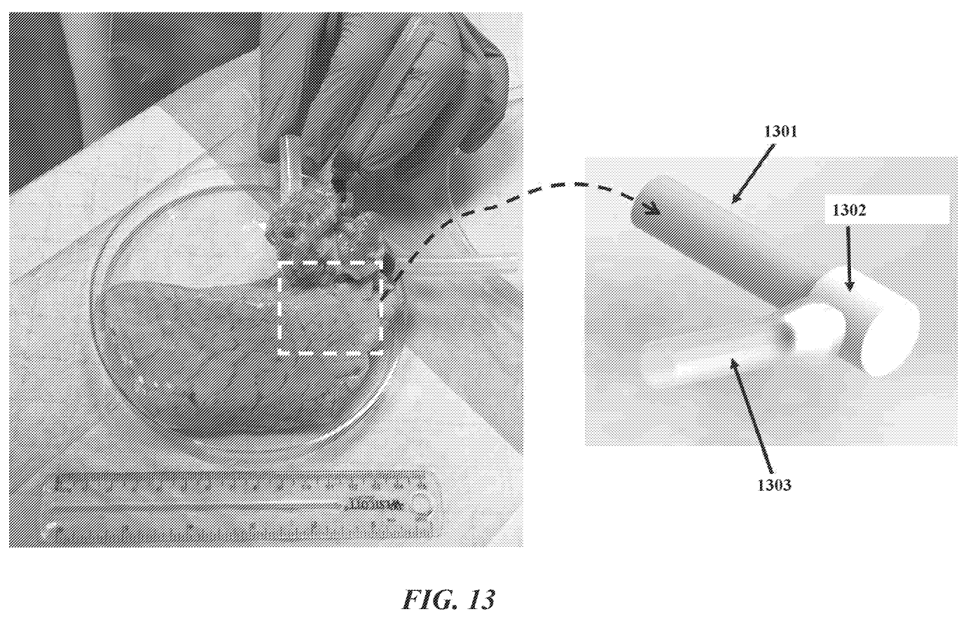

With particular reference to FIG. 13, in some embodiments a tissue and/or organ sample is introduced into tissue holder 1301 so that it can be imaged. In some embodiments, the tissue and/or organ sample present in holder 1301 is immobilized using barbed fitting 1302 so that it does not move in any direction as an ultrasound beam is passed through it. Tissue holder 1301 can also include silicon tubing 1303 which directs fluid communication between an external reservoir through a tissue and/or organ sample in tissue holder 1301 in a desired direction.

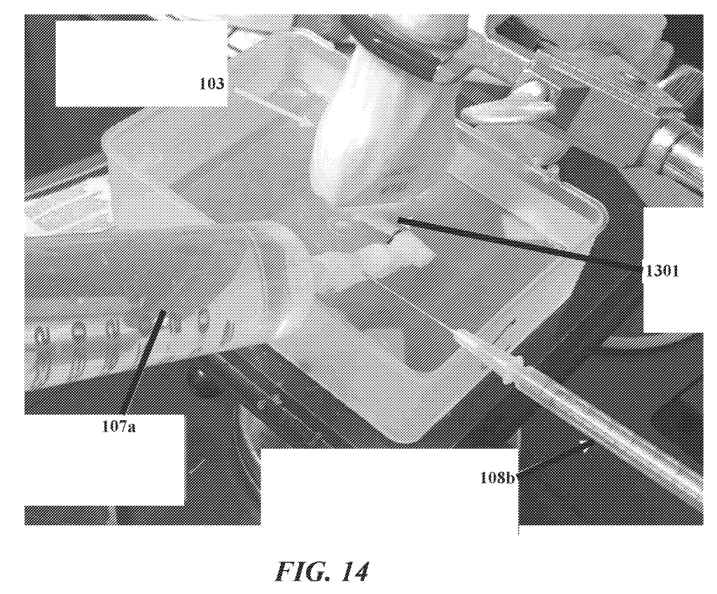

With particular reference to FIG. 14, an exemplary system of the presently disclosed subject matter can include tissue holder 1301 in which is present a tissue and/or organ sample to be imaged using probe (e.g., ultrasound transducer) 103. Fluid can be introduced through tissue holder 1301 and hence the tissue and/or organ sample via input pump 107a, which in some embodiments can be a syringe. In some embodiments, syringe pump for contrast agents 108b can be used to introduce one or more contrast agents into the fluid supplied by input pump 107a such that one or more contrast agents can perfuse the tissue and/or organ sample present in tissue holder 1301.

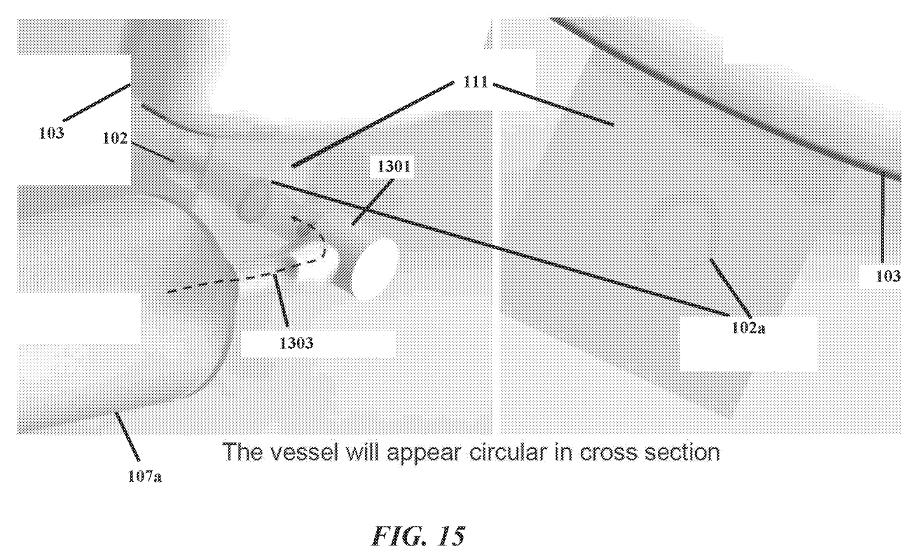

With particular reference to FIG. 15, an exemplary system of the presently disclosed subject matter can include tissue holder 1301 in which tissue 102 is present. Input pump 107a is connected to tissue holder 1301 via silicon tubing 1303 in order to introduce fluid into tissue holder 1301 and through tissue 102 (fluid flow direction indicated by the broken arrow). The tissue is imaged using probe (e.g., ultrasound transducer) 103, which directs ultrasound beam path 111 through tissue 102. In some embodiments, ultrasound beam path 111 traverses tissue 102 in a direction that is perpendicular to the axis of tissue 102. In some embodiments, tissue 102 is a blood vessel such that when ultrasound beam path 111 traverses tissue 102 in a direction that is perpendicular to the axis of tissue 102, cross-sectional image 102a of tissue 102 is captured.

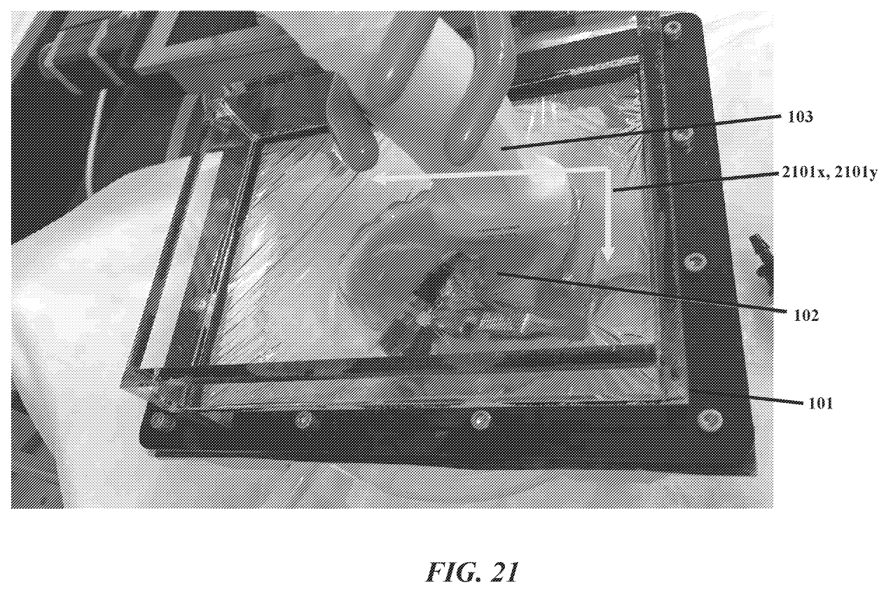

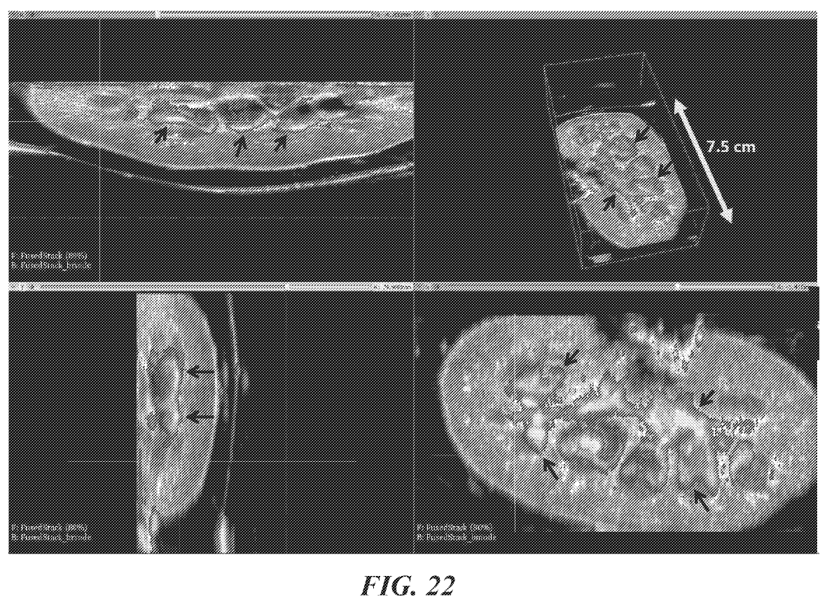

With particular reference to FIG. 21, an exemplary system of the presently disclosed subject matter can include bioreactor 101 in which tissue 102 is present in order to be imaged by probe (e.g., ultrasound transducer) 103. In some embodiments, bioreactor 101 and/or probe (e.g., ultrasound transducer) 103 can be moved with respect to each other, which in some embodiments can be in two dimensions (e.g., directions 2101x and 2101y).

The systems and methods of the presently disclosed subject matter can be employed for several different determinations of cellularity of engineered tissue scaffolds. By way of example and not limitation, the systems of the presently disclosed subject matter can be employed for determining the extent to which a tissue scaffold has been decellularized. This can be accomplished is at least two different ways.

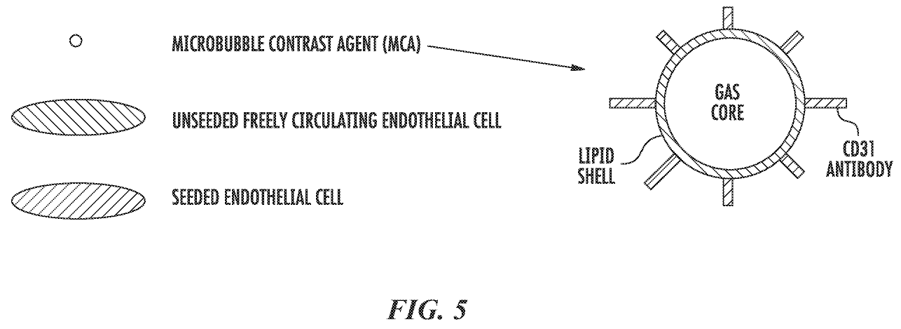

For example, in some embodiments a contrast agent that specifically binds to an endothelial cell (e.g., a microbubble that is conjugated to a ligand such as but not limited to an antibody or an antigen-binding fragment thereof that binds to CD31; see FIG. 5) can be perfused into a tissue scaffold and an extent of binding to the tissue scaffold can be assayed. In these embodiments, binding of the contrast agent to the scaffold would be indicative of endothelial cells remaining in the scaffold, which could be undesirable for a tissue scaffold that is to be recellularized with a subject's own cells prior to introducing the recellularized scaffold into the subject since the presence of non-recipient endothelial cells in the scaffold could compromise the effectiveness of the recellularized scaffold in the subject based on an induction of an anti-tissue scaffold immune response in the subject.

A second method for determining the extent to which a tissue has been effectively seeded by the desired cells comprises perfusing a contrast agent into the tissue scaffold that binds to a target present in the extracellular matrix of the scaffold, which in some embodiments can be a ligand that is only exposed to contrast agent in regions where the scaffold has been decellularized. In these embodiments, images of the scaffold should be essentially identical in appearance to b-mode images of the vessel network such that any regions of the vessel network that do not bind the contrast agent can be assumed to contain endothelial cells that block binding of the contrast agent to the extracellular matrix of the scaffold.

A further determination of cellularity of an engineered tissue (e.g., an engineered tissue scaffold and/or a bioprinted organ or tissue) that can be imaged using the systems and methods of the presently disclosed subject matter relates to determining an extent of recellularity of a tissue. In some embodiments, a decellularized scaffold is recellularized by contacting the scaffold with endothelial cells under conditions and for a time sufficient for the endothelial cells to attach to the vessels present in the scaffold. Ideally, the entire internal wall of the blood vessel should be recellularized with endothelial cells. As such, determining the extent to which a tissue scaffold is recellularized can be of value prior to introducing the engineered (i.e., recellularized) tissue scaffold into a subject.

Thus, in some embodiments the systems and methods of the presently disclosed subject matter can be designed to image tissue scaffold subsequent to recellularization. These methods can again employ contrast agents that are targeted to the extracellular matrix of a vessel to image regions of the tissue scaffold that have not been successfully recellularized, or can target an endothelial cell marker to image regions of the tissue scaffold that have been successfully recellularized.

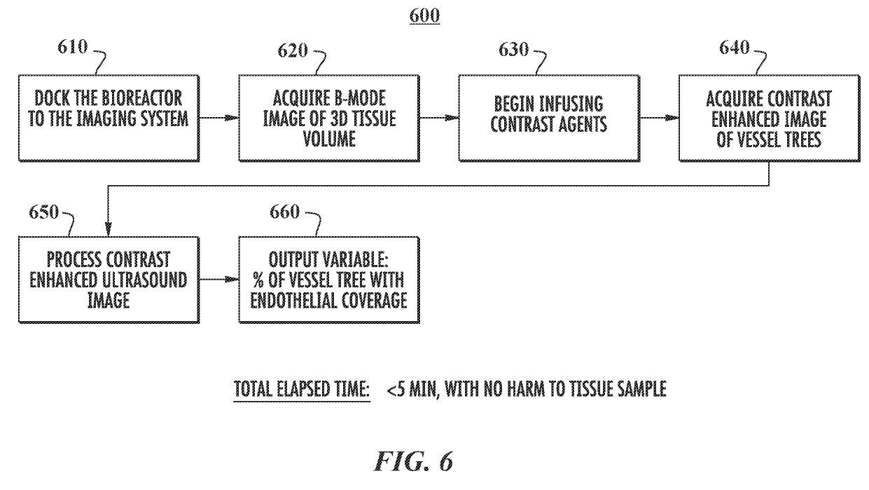

By way of example and not limitation, recellularization of a tissue scaffold present within a bioreactor can be assayed using the basic approach outlined in FIG. 6. After decellularlizing and recellularizing a tissue scaffold in a bioreactor, the extent to which the scaffold has been recellularized can be determined by docking the bioreactor to an imaging system of the presently disclosed subject matter (step 610). If desired, a b-mode image of 3D tissue volume can be determined (step 620) by imaging the scaffold in the absence of contrast agent. After an acceptable image has been obtained, one or more contrast agents can be infused into the bioreactor (step 630) by introducing the one or more contrast agents into the media perfusing the scaffold in the bioreactor. After allowing for sufficient time for the detectable moieties (e.g., a microbubble conjugated to a ligand such as but not limited to an antibody or an antigen-binding fragment or derivative thereof that binds to one of CD31, P-selectin, E-selectin, VEGF-R2, and αᵥβ₃ integrin) present with in the contrast agent solution(s) to bind to their targets (e.g., CD31, P-selectin, E-selectin, VEGF-R2, or αᵥβ₃ integrin molecules expressed by endothelial cells present within the tissue scaffold), contrast enhanced images of the vessel trees present within the scaffold can be obtained (step 640). The contrast-enhanced images (e.g., contrast enhanced ultrasound images) can then be processed by a processing unit that is a component of some embodiments of the presently disclosed subject matter (step 650). The processing unit can be programmed to accept images of the vessel trees and output various measures of cellularity of the tissue scaffold (step 660) including but not limited to a determination of the percentage of the vessel tree that has been covered by endothelial cells. In some embodiments, this noninvasive method can be accomplished in less than 5 minutes and results in no harm to the tissue scaffold while maintaining the sterility of the interior of the bioreactor (i.e., preventing contamination of the tissue scaffold itself).

In some embodiments, an extent of recellularization of a vessel lumen is determined using microbubble contrast agents (MCAs) that comprise lipid microbubbles conjugated to ligands that bind to molecules present on or in endothelial cells (referred to herein as an “endothelial cell target”; see also FIG. 5). Representative agents for providing microbubbles in vivo include but are not limited to gas-filled lipophilic or lipid-based bubbles (see e.g., U.S. Pat. Nos. 6,245,318; 6,231,834; 6,221,018; and 5,088,499; the disclosure of each of which is incorporated herein by reference in its entirety). In addition, gas or liquid can be entrapped in porous inorganic particles that facilitate microbubble release upon delivery to a subject (U.S. Pat. Nos. 6,254,852 and 5,147,631, the disclosure of each of which is incorporated herein by reference in its entirety). Such agents can be conjugated to ligands including, but not limited to peptides and antibodies or antigen-binding fragments and derivatives thereof (see e.g., U.S. Pat. No. 9,340,581, the disclosure of which is incorporated herein by reference in its entirety) that bind to endothelial cell targets such as but not limited to CD31, P-selectin, E-selectin, VEGF-R2, and αᵥβ₃ integrin.

An exemplary endothelial cell target is CD31 (also referred to as platelet and endothelial cell adhesion molecule 1), which is a member of the immunoglobulin superfamily. In humans, the CD31 protein has 738 amino acids (see Accession No. NP_000433 in the GENBANK® biosequence database; SEQ ID NO: 2) and is encoded by a 6831 nucleotide mRNA (see Accession No. NM_000442 in the GENBANK® biosequence database; SEQ ID NO: 1). Antibodies that specifically bind to the human CD31 polypeptide are available from several different commercial suppliers (e.g., Thermo Fisher Scientific Inc., Santa Cruz Biotechnology, Inc., Abcam plc.), and methods for conjugating peptides, antibodies, and/or fragments and derivatives thereof to lipids and/or microbubbles are known (see e.g., U.S. Pat. No. 9,375,397 to Bettinger et al., the disclosure of which is incorporated herein by reference in its entirety). Other exemplary endothelial cell targets include, but are not limited to P-selectin/CD62 (see Accession Nos. NM_003005 and NP_002996 in the GENBANK® biosequence database; SEQ ID NOs: 3 and 4, respectively), E-selectin (see Accession Nos. NM_000450 and NP_000441 in the GENBANK® biosequence database; SEQ ID NOs: 5 and 6, respectively), vascular endothelial growth factor receptor 2 (VEGF-R2, also referred to as kinase insert domain receptor; KDR; see Accession Nos. NM_002253 and NP_002244 in the GENBANK® biosequence database; SEQ ID NOs: 7 and 8, respectively), and the αᵥβ₃ integrin.

As used herein, the terms “antibody” and “antibodies” refer to proteins comprising one or more polypeptides substantially encoded by immunoglobulin genes or fragments of immunoglobulin genes. Immunoglobulin genes typically include the kappa (κ), lambda (λ), alpha (α), gamma (γ), delta (δ), epsilon (ε), and mu (μ) constant region genes, as well as myriad immunoglobulin variable region genes. Light chains are classified as either κ or λ. In mammals, heavy chains are classified as γ, μ, α, δ, or ε, which in turn define the immunoglobulin classes, IgG, IgM, IgA, IgD, and IgE, respectively. Other species have other light and heavy chain genes (e.g., certain avians produced what is referred to as IgY, which is an immunoglobulin type that hens deposit in the yolks of their eggs), which are similarly encompassed by the presently disclosed subject matter. In some embodiments, the term “antibody” refers to an antibody that binds specifically to an epitope that is present on an antigen expressed by an endothelial cell including, but not limited to CD31, P-selectin, E-selectin, VEGF-R2, and αᵥβ₃ integrin. In some embodiments, the term “antibody” refers to an antibody that binds specifically to CD31, P-selectin, E-selectin, VEGF-R2, or αᵥβ₃ integrin.

A typical immunoglobulin (antibody) structural unit is known to comprise a tetramer. Each tetramer is composed of two identical pairs of polypeptide chains, each pair having one “light” chain (average molecular weight of about 25 kiloDalton (kDa)) and one “heavy” chain (average molecular weight of about 50-70 kDa). The two identical pairs of polypeptide chains are held together in dimeric form by disulfide bonds that are present within the heavy chain region. The N-terminus of each chain defines a variable region of about 100 to 110 or more amino acids primarily responsible for antigen recognition. The terms variable light chain (V_{L}) and variable heavy chain (V_{H}) refer to these light and heavy chains, respectively.

Antibodies typically exist as intact immunoglobulins or as a number of well-characterized fragments that can be produced by digestion with various peptidases. For example, digestion of an antibody molecule with papain cleaves the antibody at a position N-terminal to the disulfide bonds. This produces three fragments: two identical “Fab” fragments, which have a light chain and the N-terminus of the heavy chain, and an “Fc” fragment that includes the C-terminus of the heavy chains held together by the disulfide bonds. Pepsin, on the other hand, digests an antibody C-terminal to the disulfide bond in the hinge region to produce a fragment known as the “F(ab)′₂” fragment, which is a dimer of the Fab fragments joined by the disulfide bond. The F(ab)′₂ fragment can be reduced under mild conditions to break the disulfide linkage in the hinge region, thereby converting the F(ab′)₂ dimer into two “Fab′” monomers. The Fab′ monomer is essentially an Fab fragment with part of the hinge region (see e.g., Paul (1993) Fundamental Immunology, Raven Press, New York, N.Y., United States of America, for a more detailed description of other antibody fragments). With respect to these various fragments, Fab, F(ab′)₂, and Fab′ fragments include at least one intact antigen binding domain, and thus are capable of binding to antigens.

While various antibody fragments are defined in terms of the digestion of an intact antibody, one of skill will appreciate that various of these fragments (including, but not limited to Fab′ fragments) can be synthesized de novo either chemically or by utilizing recombinant DNA methodology. Thus, the term “antibody” as used herein also includes antibody fragments either produced by the modification of whole antibodies or synthesized de novo using recombinant DNA methodologies. In some embodiments, the term “antibody” comprises a fragment that has at least one antigen binding domain.

Antibodies can be polyclonal or monoclonal. As used herein, the term “polyclonal” refers to antibodies that are derived from different antibody-producing cells (e.g., B cells) that are present together in a given collection of antibodies. Exemplary polyclonal antibodies include, but are not limited to those antibodies that bind to a particular antigen and that are found in the blood of an animal after that animal has produced an immune response against the antigen. However, it is understood that a polyclonal preparation of antibodies can also be prepared artificially by mixing at least non-identical two antibodies. Thus, polyclonal antibodies typically include different antibodies that are directed against (i.e., binds to) different epitopes (sometimes referred to as an “antigenic determinant” or just “determinant”) of any given antigen.

As used herein, the term “monoclonal” refers to a single antibody species and/or a substantially homogeneous population of a single antibody species. Stated another way, “monoclonal” refers to individual antibodies or populations of individual antibodies in which the antibodies are identical in specificity and affinity except for possible naturally occurring mutations that can be present in minor amounts. Typically, a monoclonal antibody (mAb or moAb) is generated by a single B cell or a progeny cell thereof (although the presently disclosed subject matter also encompasses “monoclonal” antibodies that are produced by molecular biological techniques as described herein). Monoclonal antibodies (mAbs or moAbs) are highly specific, typically being directed against a single antigenic site. Furthermore, in contrast to polyclonal antibody preparations, a given mAb is typically directed against a single epitope on the antigen.

In addition to their specificity, mAbs can be advantageous for some purposes in that they can be synthesized uncontaminated by other antibodies. The modifier “monoclonal” is not to be construed as requiring production of the antibody by any particular method, however. For example, in some embodiments, the mAbs of the presently disclosed subject matter are prepared using the hybridoma methodology first described by Kohler et al. (1975) Nature 256:495, and in some embodiments are made using recombinant DNA methods in bacterial or eukaryotic animal or plant cells (see e.g., U.S. Pat. No. 4,816,567, the entire contents of which are incorporated herein by reference). mAbs can also be isolated from phage antibody libraries using the techniques described in Clackson et al. (1991) Nature 352:624-628 and Marks et al. (1991) J Mol Biol 222:581-597, for example.

The antibodies, fragments, and derivatives of the presently disclosed subject matter can also include chimeric antibodies. As used herein in the context of antibodies, the term “chimeric”, and grammatical variants thereof, refers to antibody derivatives that have constant regions derived substantially or exclusively from antibody constant regions from one species and variable regions derived substantially or exclusively from the sequence of the variable region from another species. A particular kind of chimeric antibody is a “humanized” antibody, in which the antibodies are produced by substituting the complementarity determining regions (CDRs) of, for example, a mouse antibody, for the CDRs of a human antibody (see e.g., PCT International Patent Application Publication No. WO 1992/22653). Thus in some embodiments, a humanized antibody has constant regions and variable regions other than the CDRs that are derived substantially or exclusively from the corresponding human antibody regions, and CDRs that are derived substantially or exclusively from a mammal other than a human.

The antibodies, fragments, and derivatives of the presently disclosed subject matter can also be single chain antibodies and single chain antibody fragments. Single-chain antibody fragments contain amino acid sequences having at least one of the variable regions and/or CDRs of the whole antibodies described herein, but are lacking some or all of the constant domains of those antibodies. These constant domains are not necessary for antigen binding, but constitute a major portion of the structure of whole antibodies.

Single-chain antibody fragments can overcome some of the problems associated with the use of antibodies containing a part or all of a constant domain. For example, single-chain antibody fragments tend to be free of undesired interactions between biological molecules and the heavy-chain constant region, or other unwanted biological activity. Additionally, single-chain antibody fragments are considerably smaller than whole antibodies and can therefore have greater capillary permeability than whole antibodies, allowing single-chain antibody fragments to localize and bind to target antigen-binding sites more efficiently. Also, antibody fragments can be produced on a relatively large scale in prokaryotic cells, thus facilitating their production. Furthermore, the relatively small size of single-chain antibody fragments makes them less likely to provoke an immune response in a recipient than whole antibodies. The single-chain antibody fragments of the presently disclosed subject matter include, but are not limited to single chain fragment variable (scFv) antibodies and derivatives thereof such as, but not limited to tandem di-scFv, tandem tri-scFv, diabodies, and triabodies, tetrabodies, miniantibodies, and minibodies.

Fv fragments correspond to the variable fragments at the N-termini of immunoglobulin heavy and light chains. Fv fragments appear to have lower interaction energy of their two chains than Fab fragments. To stabilize the association of the V_{H} and V_{L} domains, they have been linked with peptides (see Bird et al. (1988) Science 242:423-426; Huston et al. (1988) Proc Natl Acad Sci USA 85:5879-5883), disulfide bridges (Glockshuber et al. (1990) Biochemistry 29:1362-1367), and “knob in hole” mutations (Zhu et al. (1997) Protein Sci 6:781-788). ScFv fragments can be produced by methods well known to those skilled in the art see Whitlow et al. (1991) Methods companion Methods Enzymol 2:97-105 and Huston et al. (1993) Int Rev Immunol 10:195-217.

scFv can be produced in bacterial cells such as E. coli or in eukaryotic cells. One potential disadvantage of scFv is the monovalency of the product, which can preclude an increased avidity due to polyvalent binding, and their short half-life. Attempts to overcome these problems include bivalent (scFv′)₂ produced from scFv containing an additional C-terminal cysteine by chemical coupling (Adams et al. (1993) Cancer Res 53:4026-4034; McCartney et al. (1995) Protein Fng 8:301-314) or by spontaneous site-specific dimerization of scFv containing an unpaired C-terminal cysteine residue (see Kipriyanov et al. (1995) Cell Biophys 26:187-204).

Alternatively, scFv can be forced to form multimers by shortening the peptide linker to 3 to 12 residues to form “diabodies” (see Holliger et al. (1993) Proc Natl Acad Sci USA 90:6444-6448). Reducing the linker still further can result in scFv trimers (“triabodies”; see Kortt et al. (1997) Protein Eng 10:423-433) and tetramers (“tetrabodies”; see Le Gall et al. (1999) FEBS Lett 453:164-168). Construction of bivalent scFv molecules can also be achieved by genetic fusion with protein dimerizing motifs to form “miniantibodies” (see Pack et al. (1992) Biochemistry 31:1579-1584) and “minibodies” (see Hu et al. (1996) Cancer Res 56:3055-3061). scFv-scFv tandems ((scFv)₂) can be produced by linking two scFv units by a third peptide linker (see Kurucz et al. (1995) J Immunol 154:4576-4582).

Bispecific diabodies can be produced through the non-covalent association of two single chain fusion products consisting of V_{H} domain from one antibody connected by a short linker to the V_{L} domain of another antibody (see Kipriyanov et al. (1998), Int J. Cancer 77:763-772). The stability of such bispecific diabodies can be enhanced by the introduction of disulfide bridges or “knob in hole” mutations as described hereinabove or by the formation of single chain diabodies (scDb) wherein two hybrid scFv fragments are connected through a peptide linker (see Kontermann et al. (1999) J Immunol Meth 226:179-188).

Tetravalent bispecific molecules can be produced, for example, by fusing an scFv fragment to the CH₃ domain of an IgG molecule or to a Fab fragment through the hinge region (see Coloma et al. (1997) Nature Biotechnol 15:159-163). Alternatively, tetravalent bispecific molecules have been created by the fusion of bispecific single chain diabodies (see Alt et al. (1999) FEBS Lett 454:90-94). Smaller tetravalent bispecific molecules can also be formed by the dimerization of either scFv-scFv tandems with a linker containing a helix-loop-helix motif (DiBi miniantibodies; see Muller et al. (1998) FEBS Lett 432:45-49) or a single chain molecule comprising four antibody variable domains (V_{H} and V_{L}) in an orientation preventing intramolecular pairing (tandem diabody; see Kipriyanov et al. (1999) J Mol Biol 293:41-56).

Bispecific F(ab′) fragments can be created by chemical coupling of Fab′ fragments or by heterodimerization through leucine zippers (see Shalaby et al. (1992) J Exp Med 175:217-225; Kostelny et al. (1992), J Immunol 148:1547-1553). Also available are isolated V_{H} and V_{L} domains (see U.S. Pat. Nos. 6,172,197; 6,248,516; and 6,291,158).

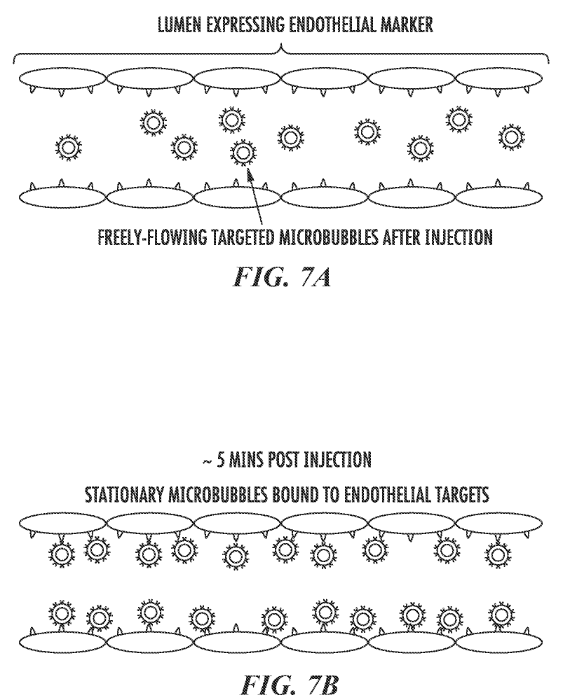

Once an appropriate contrast agent is produced, it can be introduced into a tissue scaffold present within a bioreactor for imaging. As shown in FIGS. 7A and 7B, MCAs that target endothelial markers can be employed to image cellularity of vessel trees present within engineered tissue scaffolds. The MCAs are introduced into the bioreactor and allowed to perfuse the tissue scaffold to be analyzed. In some embodiments, the system of the presently disclosed subject matter comprises a pump system that is configured to be interruptible so that the introduced MCAs can remain resident in the tissue scaffold for a desired time period before the pump system is restarted. This can increase the efficiency by which the MCAs bind to their targets in the tissue scaffold. If desired, this interruption can be combined with radiation force to promote MCA-endothelial cell interactions.

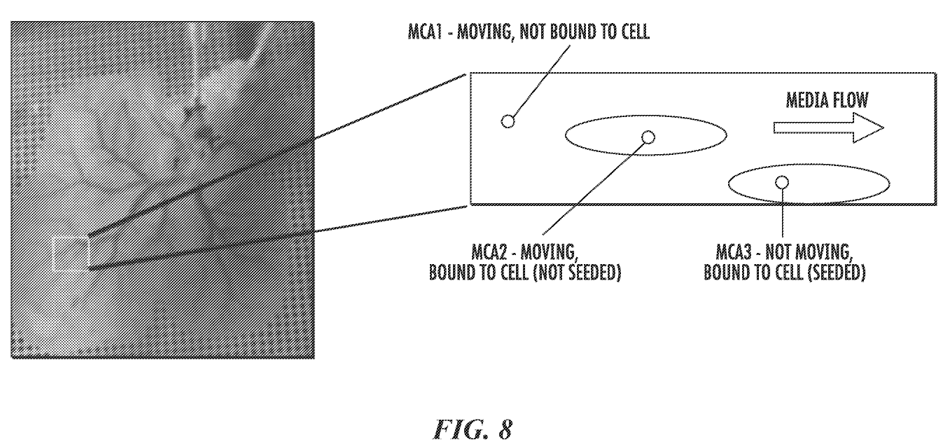

After a time sufficient to allow the MCAs to bind to their targets on endothelial cells present within the lumen of a vessel, the vessel is dynamically imaged using a system of the presently disclosed subject matter. The presently disclosed molecular imaging systems and methods delineate stationary from moving contrast agents. Those signals that are imaged as moving relate to MCAs that are unbound (see FIGS. 7A and 8), and those signals that are imaged as stationary relate to MCAs that have bound to targets (e.g., endothelial cells) present within the vessel tree (see FIGS. 7B and 8). In some embodiments, unbound contrast agents are flushed from the tissue scaffold by allowing the perfusion medium containing the contrast agent to be removed from the bioreactor, which in some embodiments can be accomplished by employing an exit line that empties to a receiver, the contents of which is not recirculated to the bioreactor. In such an embodiment, at least one of the input lines must be in fluid communication with fresh medium such that perfusion of the tissue sample with medium is maintained despite continuous removal of the medium from the system via the exit line(s).

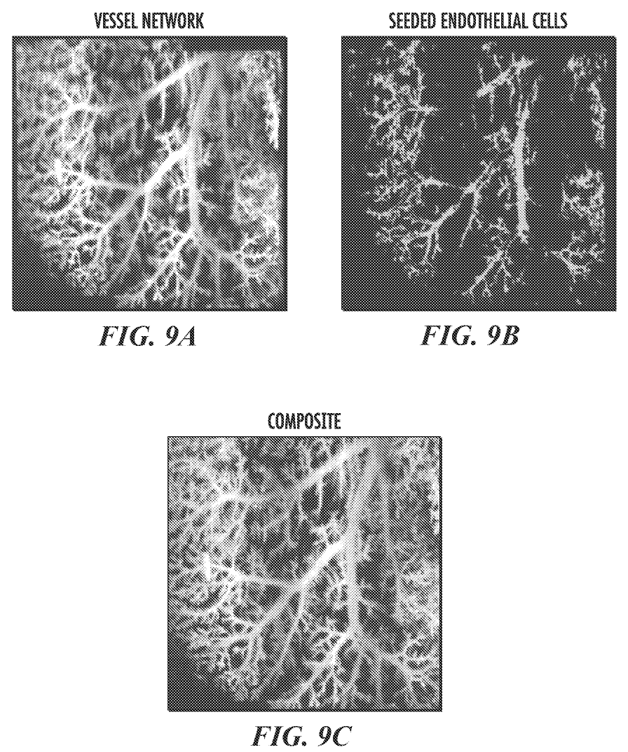

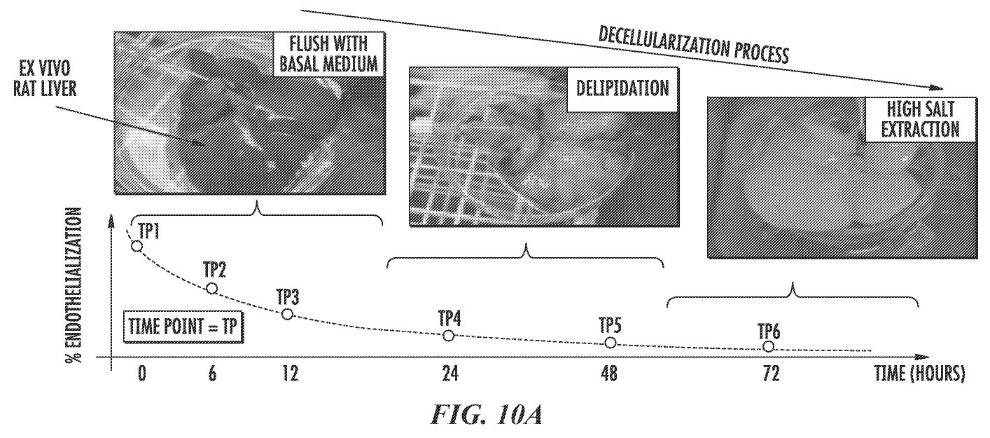

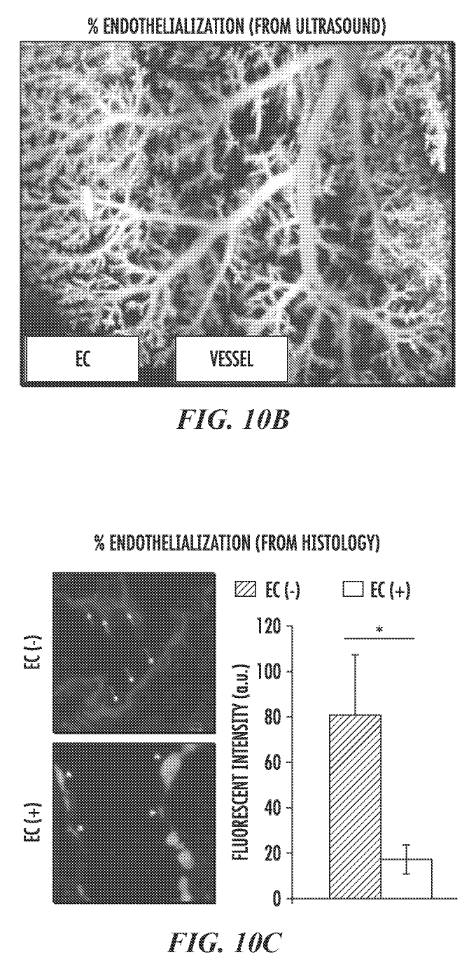

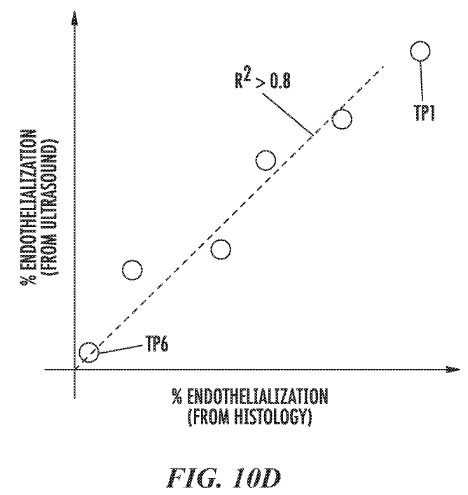

Thus, the systems and methods of the presently disclosed subject matter can be employed to determine an extent of recellularization of a vessel network present in an engineered tissue scaffold. By way of example and not limitation, FIGS. 9A-9C provide examples of outputs that can be generated by the systems of the presently disclosed subject matter. In FIG. 9A, the vessel network is visualized via a b-mode image taken prior to the introduction of any contrast agents into the engineered tissue scaffold. After a contrast agent has been introduced, signal derived from contrast agent interactions with endothelial cells present in the vessel network can also be imaged as shown in FIG. 9B. In some embodiments, the signal derived from contrast agent interactions with endothelial cells present in the vessel network is depicted in a color different from that of the b-mode image, such that a composite of the b-mode image and the signal derived from contrast agent interactions with endothelial cells present in the vessel network can be generated (see FIGS. 9C and 10B), thereby permitting a visual representation of the extent of recellularization of a vessel network present in an engineered tissue scaffold. In some embodiments, the stationary signal data entirely align with the 3D vessel data (e.g., b-mode data), which means that the entire vessel network has endothelial cell coverage.

In some embodiments, the presently disclosed subject matter also encompasses one or more secondary bypass circuits that can be employed for removing contrast agents from the bioreactor. Such a design is depicted in FIG. 11.

As set forth previously, the decellularization of a tissue scaffold can also be visualized over time using the systems and methods of the presently disclosed subject matter. An exemplary approach to decellularization analysis is depicted in FIG. 10A. As shown in FIG. 10A, an ex viv tissue sample can be obtained and flushed with basal medium. The tissue sample can be delipidized and extracted with high salt to decellularize the tissue sample. As such, this process can take place over several days, and at various time points during the process (TP1-TP6 in FIG. 10A), the percent endothelialization remaining in the tissue sample can be assayed using the systems and methods of the presently disclosed subject matter. FIG. 10A depicts an exemplary decellularization time course in which the percent endothelialization of the tissue sample would be 100% at TP1 (e.g., t=0 hours), and would decrease over time as the tissue sample becomes increasingly decellularized (e.g., TP2-TP5). After appropriate delipidation and extraction with high salt, the tissue sample would be sufficiently decellularized to be used as a tissue scaffold for recellularization (e.g., TP6). FIGS. 10B and 10C illustrate how endothelialization (“seeding”) can be quantified via ultrasound and histology, respectively. The vessel images in FIG. 10B include endothelial mapping simulated on top. FIG. 10D is a graph of simulated data illustrating a correlation of greater than 0.8 between the non-invasive ultrasound of the presently disclosed subject matter and the gold standard histological analysis for the six (6) time points shown in FIG. 10A.

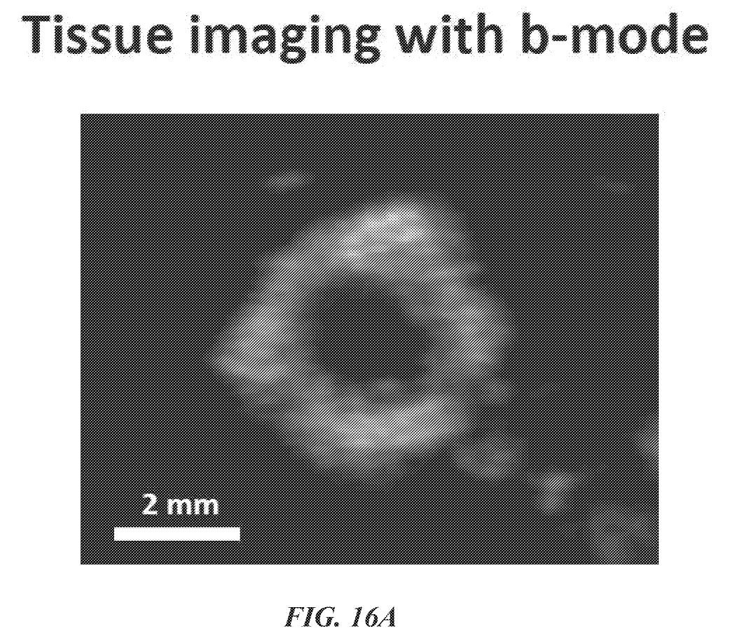

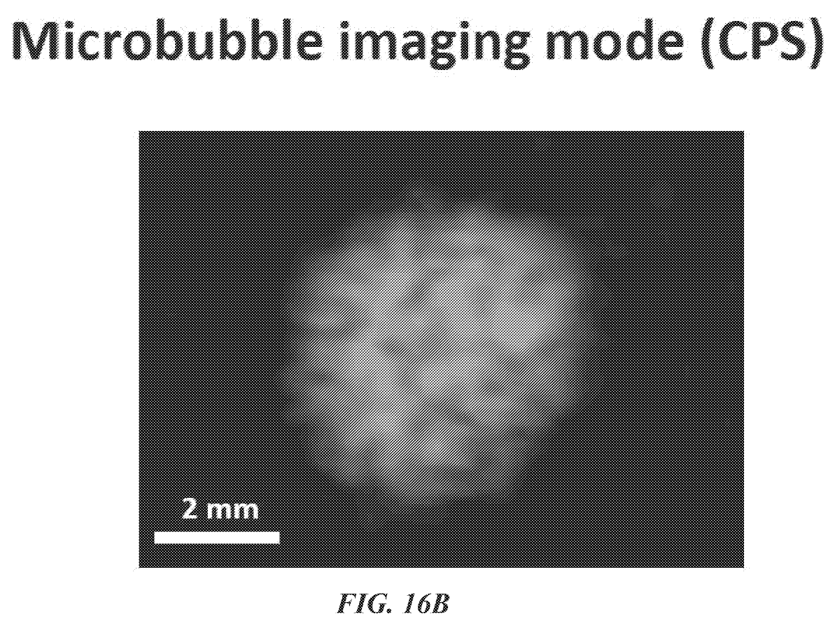

FIGS. 16A and 16B show representative examples of tissue imaging using an imaging system of the presently disclosed subject matter. FIG. 16A depicts b-mode imaging of the tissue in the absence of contrast agent for delineating the borders of a tissue sample. FIG. 16B is an image after introduction of a microbubble contrast agent (MCA). The introduction of a contrast agent into the tissue allows for imaging of the contrast agent within the lumen of the tissue as well as bound to targets (e.g., endothelial cells) present therein. For microbubbles to be delineated from tissue, in some embodiments a contrast-specific mode (e.g., Cadence Pulse Sequence; CPS) can be employed (see FIG. 16B).

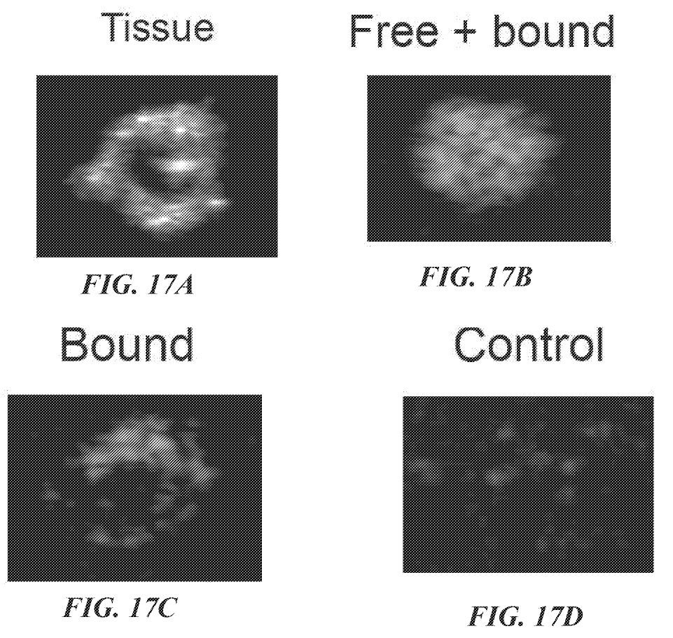

FIGS. 17A-17D depict four (4) representative images of a vessel imaged with the imaging system of the presently disclosed subject matter. FIG. 17A is a b-mode image that shows a cross section of the vessel. The vessel lumen predictably shows an absence of signal (i.e., is largely black). FIG. 17B is an image of the vessel when fully perfused with targeted MCAs of the presently disclosed subject matter. The targeted MCAs perfuse the vessel lumen. FIG. 17C is an image of the vessel after a saline flush to remove unbound MCAs. The lumen of the vessel has been cleared of unbound microbubbles, whereas targeted MCAs are bound to the endothelial cells of the vessel wall. FIG. 17D is an image showing the vessel after the targeted MCAs were destroyed. As shown, there was some ambient signal from bubbles in the media but that it was substantially less than that produced by the targeted microbubbles when bound to their cellular targets on the wall of the vessel.

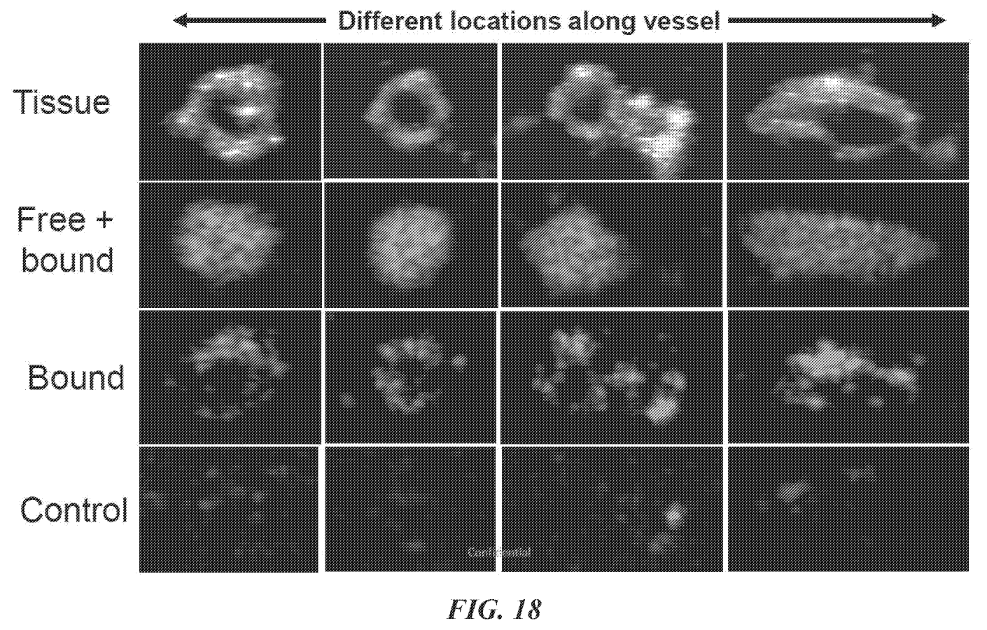

FIG. 18 is a series of images similar to those depicted in FIG. 17 taken at four (4) different locations along the length of the vessel. From top to bottom, the groups of images are b-mode images of the tissue, images that show free and bound MCAs, images that show bound MCAs after removal of unbound MCAs, and controls after the targeted microbubbles present in the MCAs were destroyed. This Figure shows that the images are consistent along the vessel's length, demonstrating that flow of the MCA along the vessel efficiently labels the vessel along its length for imaging.

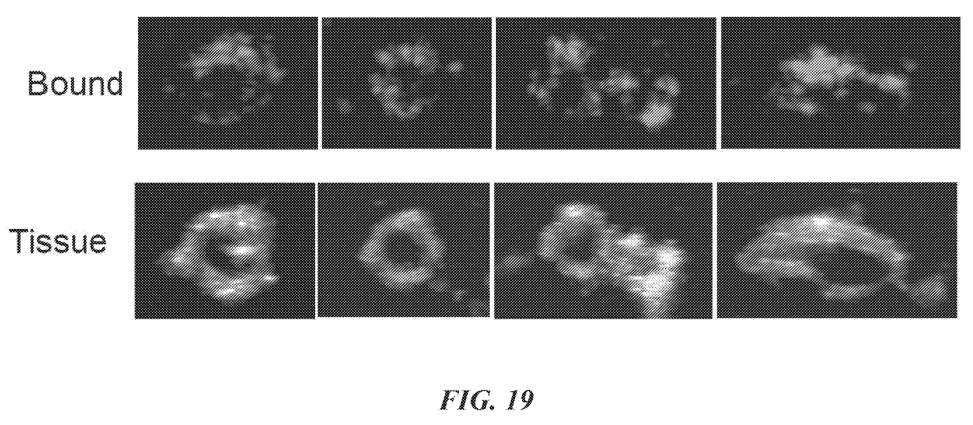

FIG. 19 is a juxtaposition of rows 3 (top panel) and 1 (bottom panel) of FIG. 18, highlighting the efficiency at which endothelial cells along pulmonary vessel walls can be imaged using the 3D non-invasive visualization systems of the presently disclosed subject matter.

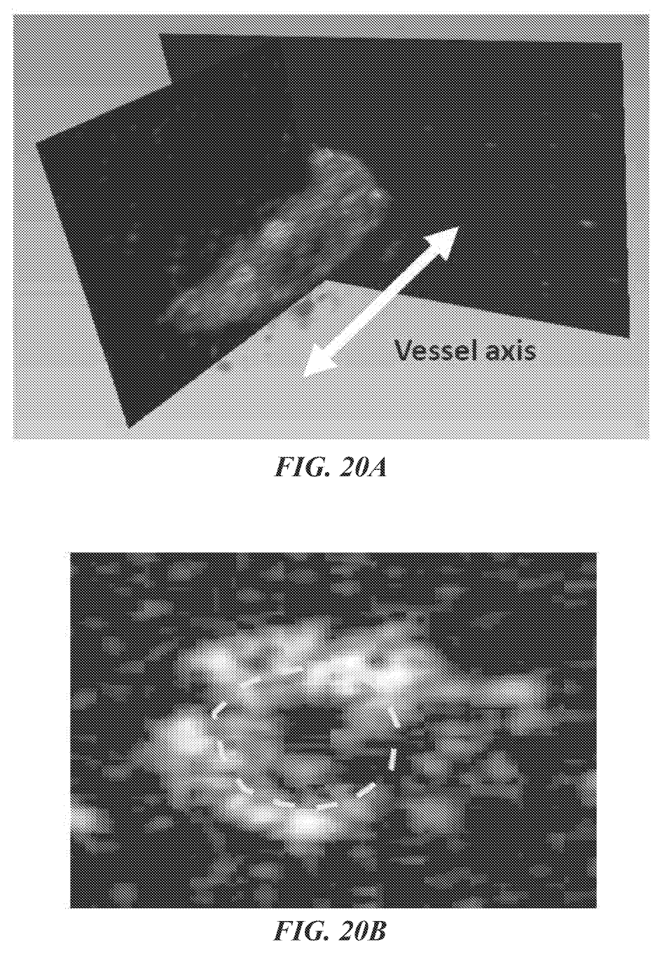

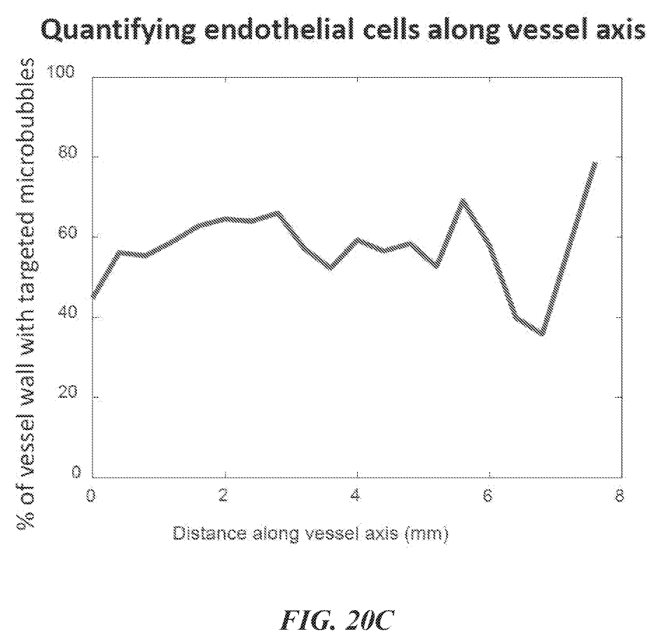

FIGS. 20A-20C present another example of a vessel imaged with the imaging system of the presently disclosed subject matter. FIG. 20A presents a three-dimensional image of a vessel that can be produced from data acquired by an imaging system of the presently disclosed subject matter using a microbubble contrast agent (MCA) that binds to targets present on the inner luminal surface of the vessel. FIG. 20B is a cross sectional representation of the same vessel depicted in FIG. 20A. The dashed line is indicative of the inner luminal surface. FIG. 20C is a graph of distance along a vessel axis in millimeters from an arbitrary start point versus the percent of the vessel wall that to which targeted microbubbles have bound. As can be seen from FIG. 20C, a fairly consistent level of cellular distribution was observed over the measured length of the vessel.

Other parameters of interest of engineered tissue scaffolds can also be determined using the systems and methods of the presently disclosed subject matter. For example, tissue stiffness can be assayed using acoustic radiation force imaging, vascular network patency can be assayed with acoustic angiography and/or Doppler techniques, tissue oxygenation can be assayed using photoacoustics, nanoparticles targeted to extraluminal targets can be imaged with photoacoustics, and stem cell tracking can be accomplished using functional optimal imaging including, but not limited to bioluminescence imaging (BLI) and fluorescence imagine (FLI).

The systems and methods of the presently disclosed subject matter can provide various advantages over currently employed techniques for visualizing cellularization of engineered tissue scaffolds (in some embodiments, recellularization of engineered tissue scaffolds). In particular, current visualization techniques typically require destruction of the scaffold in order to properly visualize the extent to which the scaffold has become cellularized or in some embodiments recellularized. By way of example and not limitation, comparing an exemplary embodiment of the presently disclosed subject matter (referred to herein as “OrganVis”) to current destructive visualization techniques with respect to the time required to determine that a particular cellularization/recellularization effort had succeeded or failed and the costs associated with such a failed study, it would require less than two (2) days to determine that a particular cellularization/recellularization effort failed (i.e., that a tissue scaffold was inadequately seeded) using the exemplary embodiment of the presently disclosed subject matter (OrganVis). Using current destructive techniques, which require that the engineered tissue scaffold be manipulated physically in order to assess cellularization/recellularization, the same determination would require greater than eight (8) days. The associated costs would also be greatly reduced (e.g., reduced by approximately 50%) using the exemplary embodiment of the presently disclosed subject matter (i.e., the OrganVis), primarily due to reduced reagent usage in the non-destructive visualization technique of the presently disclosed subject matter as compared to destructive visualization techniques currently required. Furthermore, unlike current destructive visualization techniques, the sample analyzed using the exemplary embodiment of the presently disclosed subject matter is not destroyed by the presently disclosed visualization techniques, meaning that the same sample can be further seeded and/or re-seeded and analyzed thereafter without the requirement of providing a new scaffold, thereby resulting in a significant saving of scarce resources (i.e., the scaffolds).

In some embodiments, the presently disclosed subject matter can be employed for imaging and analysis of engineered organs and tissues that are produced using a three-dimensional printer. By way of example and not limitation, U.S. Pat. No. 7,051,654 to Boland et al., entitled “Ink-jet printing of viable cells”, U.S. Patent Application Publication No. 2011/0250688 of Hasan entitled “Three Dimensional Tissue Generation”, and U.S. Patent Application Publication No. 2017/0198252 of Mironov et al. entitled “Device and Methods for Printing Biological Tissues and Organs”, each of which is incorporated herein by reference in its entirety, describe devices and methods that can be employed for producing biological materials such as engineered tissues and organs. In whatever way that the engineered tissues and organs are generated, however, there would still be a need to analyze the extent to which the engineered tissues and organs have been properly seeded with, for example, endothelial cells and/or vascularized. These assessments, including but not limited to confirming that an acceptable degree of seeding/reseeding and/or vascularization had occurred, can be performed using the devices and methods of the presently disclosed subject matter.

In some embodiments, the engineered tissue sample is a 3D printed organ or tissue. With particular reference to FIG. 12, the bioreactor can be a sealed printing chamber in which an organ or tissue is printed via a biomaterial printing head, optionally a biomaterial printing head under robotic control. FIG. 12A depicts an embodiment of the presently disclosed subject matter in which the imaging probe is physically affixed to a robotically controlled printing head of a 3D printing device adapted to produce an engineered organ or tissue. In such an arrangement, the imaging probe spatially tracks the motion of the robotically controlled printing head of the 3D printing device in three dimensions and can thus provide real time imaging of the printing of the engineered organ or tissue sample as the printing process proceeds. FIG. 12B depicts an embodiment of the presently disclosed subject matter in which the imaging system is not fixed to the robotically controlled printing head of the 3D printing device adapted to produce the engineered organ or tissue, but rather is placed at a location adjacent to (in some embodiments laterally and in some embodiments beneath) the engineered organ or tissue in order to image the engineered organ or tissue. Where the imaging system is not fixed to the robotically controlled printing head of the 3D printing device, the imaging system can be placed in any position where the desired imaging of an appropriate region of the engineered organ or tissue can be performed. Alternatively or in addition, the imaging system can be adapted to rotate around the engineered organ or tissue to image several different regions of the same and/or from several different spatial locations before, during, and/or after the printing process in order to monitor the progress of the printing process and/or ascertain whether or not the printing process proceeded to the extent desired.

### EXAMPLES

The following Examples provide further illustrative embodiments. In light of the present disclosure and the general level of skill in the art, those of skill will appreciate that the following EXAMPLES are intended to be exemplary only and that numerous changes, modifications, and alterations can be employed without departing from the scope of the presently disclosed subject matter.

### Background of the Examples

Automated Whole-Organ Construct Imaging.

Ultrasound imaging poses a number of advantages compared to other imaging modalities that fit the niche of whole-organ in-bioreactor imaging. First, ultrasound is a highly-portable technology. In some embodiments, portability is a desirable feature for bioreactor imaging because bioreactors themselves are non-portable. Cultured organs and tissues are extremely sensitive. They must be kept sterile, must constantly be perfused to facilitate oxygen transport and nutrient/waste exchange, and must remain inside an incubator where the environment can be maintained within specified parameters. Therefore, in some embodiments the noninvasive imaging systems of the presently disclosed subject matter are designed to be mobile so that they can be moved to the bioreactor as opposed to a paradigm where the bioreactor or the organ/tissue is moved to the imaging system.

Ultrasound is also characterized by the advantage that it utilizes nonionizing radiation and has fewer potential side effects compared to X-ray technologies such as CT. Ionizing radiation can be tolerated by living organisms, but organoids developing in bioreactors typically lack mechanisms to repair DNA damage caused by ionizing radiation.

Additionally, ultrasound imaging has the tomographic depth of penetration necessary to image human-sized organs. Human livers can easily exceed thicknesses of 5 cm, which precludes the use of many optical imaging techniques that are depth limited such as Optical Coherence Tomography (OCT). Therefore, ultrasound represents an adaptable solution for in-bioreactor whole-organ/tissue imaging.

Despite the many benefits of ultrasound imaging, one of the challenges is the potential for large inter-user and intra-user variability. This variability arises from the handheld nature of the modality and the requirement that a human operator properly place the ultrasound transducer. Additionally, conventional ultrasound imaging is largely limited to 2D images. Transducers to produce 3D ultrasound images exist, but they require complicated matrix array technology that can be very costly. Combining ultrasound with simple robotics as presently disclosed can alleviate these two challenges by: (1) ensuring probe placement is consistent; and (2) allowing three-dimensional ultrasound acquisitions by scanning a 2D probe in the third dimension. An added benefit is that a fully automated robotic system obviates the need for a trained sonographer, reducing the overhead and overall expense of imaging. With the presently disclosed systems, a biologist is able to capture a full 3D representation of a developing organ and/or engineered tissue without having to sacrifice the organ for histology.

Ultrasound-Compatible Bioreactor that Maintains Sterility in Longitudinal Studies.

A unique component of the presently disclosed subject matter is the development of an ultrasound-compatible bioreactor that can maintain sterility. Current bioreactor chambers are typically made of glass or thick plastic containers that are sterilizable via autoclaving and sealed to the outside environment. The presently disclosed subject matter, on the other hand, comprises in some embodiments an ultrasonically compatible bioreactor intentionally designed for maximum ultrasound penetration. To achieve this goal, an acoustic window made of thin-film plastic is constructed into the base of a conventional bioreactor design using autoclavable materials and sealants. Ultrasound imaging proceeds in some embodiments in a bottom-up approach through this membrane. With this new bioreactor design, ultrasound is able to “see through the walls” of the bioreactor and image the contents without compromising the sterility of the bioreactor contents. In addition to the presently disclosed bioreactor design, the presently disclosed systems in some embodiments comprise a support scaffold to offset the bioreactor from the shelf floor in order to provide a docking space for the presently disclosed imaging unit.

Mobile Imaging Station with Bioreactor Docking Mechanism.

As disclosed herein, the presently disclosed imaging technology is brought to the bioreactor, which in some embodiments cannot or should not leave the incubator. Therefore, in some embodiments the presently disclosed subject matter provides a cart-based ultrasound system and a docking mechanism that couples the imaging hardware to the bottom of the bioreactor within the incubator. Like conventional scanners, in some embodiments the major components of the ultrasound engine reside on the cart, while in some embodiments the imaging transducer and robotics reside on a separate component that couples to the cart with a cable and/or a robotic arm.

In some embodiments, an exemplary imaging workflow proceed as follows: a user positions the cart-based ultrasound system in proximity to an incubator in which one or more bioreactors are present, and places the mobile arm containing the imaging transducer under the desired bioreactor(s) and in proximity to one or more acoustically transmissible regions (e.g., windows) of the bioreactor(s). Imaging proceeds with user input via a computer console component of the cart-based system. Upon completion, the imaging unit is undocked from the bioreactor(s) and returned to the cart. The cart and docking mechanism is designed with flexibility in mind, including the ability to dock the imaging unit to bioreactor(s) at varying heights, and in any brand of incubator.

High-Resolution Microvascular Imaging with Annular Arrays.

An Acoustic Angiography imaging technology available from SonoVol of Durham, N.C. is the highest-resolution ultrasound vessel mapping technique available (see U.S. patent application Ser. No. 13/393,500, published as U.S. Patent Application Publication No. 2012/0220869, now U.S. Pat. No. ______, incorporated by reference in its entirety). The SonoVol Acoustic Angiography imaging technology is deployed in a new commercial context by providing the core framework on which the presently disclosed organ analysis workflow is built. These 3D microvascular images are capable of illustrating vascular channels within a tissue scaffold that are perfused with 100 μm resolution. When microvessel images are used in conjunction with the presently disclosed ultrasonic molecular imaging, the system produces the previously unavailable but critical metric of “percent endothelial coverage”.

Modifications of the SonoVol Acoustic Angiography imaging technology include the use of annular arrays that provide radial beam symmetry with relatively few elements, improved signal to noise ratios (SNR) and depth of field (DOF), and enhanced lateral resolution over the DOF. Annular arrays have the simplicity of single-element systems, yet have image quality better than an equivalent linear-array system. This imaging performance derives from the large aperture and ability to axially focus the annular array over a broad DOF. A linear array has a smaller transmit aperture (less energy transmission) and a non-symmetric beam that has an out-of-plane beamwidth greater than the in-plane beamwidth.

Cell Tracking Using Ultrasound Molecular Imaging.

This technology employs microbubble contrast agents bearing a protein, a peptide, and/or an antibody targeted to a specific cellular marker. Increased ultrasound signal from the accumulation of the injected contrast agent enables spatial localization of increased expression levels of the target. For example, cRGD peptide-loaded microbubbles will target to αᵥβ₃ integrins expressed by angiogenic cells (a method for tumor imaging). In some embodiments of the presently disclosed subject matter, CD31 antibodies are used to target endothelial cells, allowing the quantity of stationary signals (i.e., effectively seeded endothelial cells) to be determined relative to the total surface area of a microvascular network. Microbubbles have been conjugated to CD31 and successfully targeted to endothelial cells for in vitro studies of sonoporation therapeutics (Kooiman et al. (2011) 154 J Control Release 35-41), although they have not been employed in a manner similar to that disclosed herein.

Image Registration and Serial Analysis Software.

Neurosurgeon Dr. Elizabeth Bullitt of the University of North Carolina pioneered methods for extracting blood vessels from 3D MRI images, and correlating the same to underlying disease states. These algorithms have been patented (see U.S. Pat. Nos. 8,090,164 and 8,233,681, the disclosure of each of which is incorporated herein by reference in its entirety) and modified by the inventors of the presently disclosed subject matter for use in high-resolution Acoustic Angiography images. For example, beyond simply quantifying the patency and vessel architecture within individual scaffolds, the presently disclosed platform can use these vessel maps as anchor points to align images acquired at different timepoints. This provides for spatial-temporal analyses of cell seeding, which is a weakness of conventional histological analyses of engineered tissues.

Thus, in some embodiments the presently disclosed subject matter ensures that non-invasive measurements of cells within scaffolds accurately correlate with conventional histopathological assessments, and that the measurement approach itself does not compromise the integrity of the organ sample.

Thereafter, several further investigations are undertaken. First, a formulation of contrast agent(s) that minimizes expense to the user but achieves sufficient measurement accuracy is identified. Next, the hardware components are deployed within a mobile cart based form factor which allows it to be shared between labs and requires no physical manipulation of the bioreactor. Third, longitudinal and intra-user consistency is validated. And fourth, that the platform has utility in analyzing human-sized organs is confirmed.

### Example 1

### Ultrasound can be Used to Image Decellularized Liver Scaffolds

Livers were harvested from Wistar rats and decellularized following standard techniques. Multiple imaging protocols were tested on decellularized scaffolds including: flash replenishment imaging using an Acuson Sequoia 512 (Siemens Medical Solutions USA Inc, Mountain View, Calif.) and 15L8 transducer to measure perfusion time; acoustic angiography using a Visualsonics Vevo770 (Toronto. Ontario, Canada) and prototype dual-frequency transducer to obtain vessel morphology maps; and high-resolution B-mode imaging at 30 MHz with a Vevo770 for anatomical images. All three imaging modes were performed with ultrasound transducers coupled to linear motion stages to capture 3D volumetric data. Acoustic angiography and perfusion imaging revealed patent vasculature in the scaffold as evidenced by the delayed peak time of the organ perfusion curve, thereby demonstrating the power of noninvasive imaging of organ constructs.

### Example 2

### Targeted Microbubbles can be Used to Visualize Specific Cells in 3D

Following intravenous injection, molecularly targeted microbubbles bearing one or more targeting ligands circulate through the vasculature and eventually accumulate in regions expressing the target molecules. These areas are depicted on ultrasound data as bright regions locating the molecules of interest. Targeted microbubbles have recently been combined with acoustic angiography imaging to significantly improve contrast-to-tissue ratio (CTR) of molecular images from 0.53 dB to 13.98 dB. Given these results taken in vivo, molecular sensitivity is predicted to be even greater in a bioreactor, with less attenuation, tissue motion, and dose limitations.

### Example 3

### Design and Test of Bioreactor with Acoustic Window(s) for Non-Invasive Imaging

Bioreactors have conventionally been fabricated out of heavy plastic or glass, which preclude ultrasound imaging through the bioreactor walls. This is due to strong reflection coefficients of these materials. Therefore, a bioreactor chamber with an acoustically transparent window that allows ultrasound waves to penetrate into the media is provided. Particularly, a novel bioreactor design with an acoustically transparent floor made of thin, ultrasound-amenable material is produced and tested. The design is such that ultrasound penetrates into the bioreactor without the chamber having to be opened (thus preserving sterility), while maintaining all functionality of a traditional bioreactor including the ability to be sterilized with an autoclave.

Bioreactor Chamber.

The bioreactor chamber is constructed following a series of steps. First, a 0.125″ thick polycarbonate tube is chemically bonded to a flat circular polycarbonate sheet with a rectangular opening cut from the center. Additionally, input and output ports for perfusion tubing are drilled into the side of the polycarbonate tube, and barbed tubing connectors are epoxied into the holes. To form the ultrasonically transparent window, a sheet of polymethylpentene with a thickness of 76 μm is stretched over the opening and bonded to the polycarbonate using a cyanoacrylate adhesive (Prism 405, LOCTITE™, Henkel Corp., Cary, N.C.). A circular polycarbonate flange is bonded to the outer top of the tube. Finally, the lid of the bioreactor is constructed from a second sheet of polycarbonate with a matching diameter to the flange. Silicone rubber is cut to match the flanged perimeter of the top of the bioreactor, and holes are drilled and tapped that allow the lid to be sealed shut with screws. Tightness of seals is tested by filling the bioreactor with water and ensuring that no leaks form over the course of 24 hours.

Fluid Circuit.

Because the presently disclosed approach requires the introduction of targeted ultrasound contrast agents, the hardware setup is slightly more complex than a standard perfusion bioreactor. Quantifying molecularly targeted microbubbles within ultrasound data requires the delineation of those which have bound to their stationary targets from those which are freely circulating. In vivo studies with molecularly targeted agents typically resolve this issue by waiting a period of 5 to 15 minutes before imaging the targeted bubbles, during which time freely circulating contrast agents are cleared from the body through exhalation of the gas through the lungs and clearance through the reticuloendothelial system. Because an organ within a bioreactor exists in isolation, this waiting period could be substantially longer and highly inconvenient without a method to actively remove freely circulating contrast agents.

To address this, FIG. 11B depicts a secondary bypass circuit that can be activated to rapidly clear the bolus of freely circulating bubbles after they make their first pass through the organ without modifying the total volume of media within the system. The output from the organ can be directed to a waste container during this time, while a reservoir of fresh sterile media can be introduced to replace the extracted volume. The bypass circuit is deactivated when the contrast agent bolus has passed through the organ. Both Primary and Secondary circuits are made from tubing with flow driven by peristaltic pumps. A silicone injection port is positioned in line with the input portion to the tissue to allow contrast agents to be introduced via a sterile syringe. Downstream contrast agents are monitored with a specialized medical grade bubble detection sensor to determine the timing of Primary vs. Secondary circuit transition.

Confirmation that Bioreactor can be Used for Longitudinal Imaging Studies without Loss of Sterility.

After the bioreactor is constructed, its capacity to be sterilized and then imaged with the presently disclosed system without loss of sterility is tested. An additional four identical bioreactors are constructed to ensure reproducibility. Each bioreactor is placed in an autoclave programmed with a standard decontamination protocol (120 minutes, 121° C., 15 psi). Bioreactors are then transferred to a biologic hood and prepared identically as though a scaffold was to be placed within it for a recellularization procedure, though for these studies no scaffold will be included. The circulation pumps is set up to circulate media through the chamber. Over the course of one week, the bioreactors are imaged a total of five times. During the imaging studies MCAs are introduced into the system as they would be in a scaffold imaging study. At the conclusion of the five-day study, the fluid within the bioreactor is tested for contamination by collecting a 0.5 mL sample of media from each and allowing it to incubate at 37° C. on a preparation of sterile agar gelatin for another 72 hours. These samples are controlled by an additional agar plate prepared with the same formulation but not washed in bioreactor media. Contamination is evaluated by counting the number of bacterial colony forming units (CFUs) on the agar plates relative to the control plate.

Validate Cell Tracking Imaging Protocol in Phantom.

The presently disclosed imaging approach for mapping seeded cells relies on determining where stationary microbubble contrast agent signals are within an actively perfused 3D vascular network. This is because a stationary microbubble indicates a targeting event has occurred between a microbubble and seeded cell. To map these stationary bubbles, the acquisition protocol proceeds via the following steps:
1. Acquire 3D B-mode image to provide anatomical context for vascular image data;
2. Begin infusion of targeted microbubbles via calibrated syringe pump;
3. When microbubbles are detected at the output of the organ, turn off the primary infusion pump;
4. Allow targeted microbubbles an incubation period within scaffold to promote binding to cellular targets;
5. Switch bypass valve to the Secondary perfusion circuit and activate pump #2 to clear the non-targeted bubbles from interior of organ. The in-line microbubble detector can be used to determine when 95% of the bolus has passed through the organ. When finished, switch bypass valve to the Primary perfusion circuit;
6. Acquire 3D Acoustic Angiography image of scaffold. Signal detected from any non-targeted bubbles that remain can be suppressed using signal processing techniques; and
7. If an image of the vessel network is desired, an additional dose of contrast agents can be introduced, and the organ scanned again in Acoustic Angiography mode. This allows the user to quantify seeded cells as a percentage of vessel network length (vs. overall tissue volume).

This protocol is evaluated in a phantom prior to an ex vivo organ scaffold. The phantom is positioned within the bioreactor. Briefly, the phantom is made from a gelatin mixture which includes biotin particles. A channel through the interior of the phantom is created which allows contrast to flow through its interior. If contrast agents are conjugated to avidin prior to infusion, they bind to the biotin molecules along the phantom wall. A total of ten phantoms are prepared with different concentrations of biotin between 0 and 10% by mass. A scanner that allows a transducer to be raster scanned beneath a target, using a coupling bath to ensure artifact-free imaging is employed. Criteria for success are an R²≥0.8 for targeted avidin bubble signal correlation with biotin concentration in phantom across 10 concentrations.

Criteria for success of the overall study are that all seals of the bioreactor remain watertight following both construction and autoclaving. No bacterial growth is observed in any of the bioreactors following the multi-imaging study. R²≥0.8 for biotin concentration with ultrasound signal.

### Example 4

### Develop and Test Targeted Microbubbles that can Selectively Bind to Endothelial Cells

While endothelial cells are certainly not the only important cell within organ scaffolds, they are a critical component to creating a biocompatible implantable organ. This EXAMPLE utilizes an in vitro cell culture assay to validate molecularly targeted contrast agents that bind to these cells. In this aim, a size selected contrast agent is formulated and validated with respect to adhesion to endothelial cells.

Creation of Size-Sorted Targeted Microbubble Formulations.

Most commercially available microbubble formulations are polydisperse in size, with contrast agent diameter distributions spanning a range of <1 μm to >10 μm. However, some commercial suppliers can provide a size-sorting microbubble formulation approach. Size sorted microbubbles can produce over a 1,000-fold improvement in microbubble acoustic response compared to conventional polydisperse contrast agents. This allows for less antibody per injection, which is a driving cost for molecular imaging contrast agents, and still achieves a sufficient ultrasound imaging response to understand cell distribution in scaffolds. Additionally, instead of using avidin-biotin chemistry, maleimide-thiol chemistry is employed to bind the CD31-antibody to the contrast agent shell.

Maleimide-thiol contrast agents are formulated similarly to those described in Anderson et al. (2010) 45 Invest Radiol 579-585 (incorporated herein by reference in its entirety). This reference described targeting tumor angiogenesis via the VEGFR-2 marker, whereas the presently disclosed subject matter employs a CD31 antibody conjugated to a thiol group (Thermo Fisher). The Advanced Mircrobubble Labs team provides size sorted maleimide bearing contrast agents with mean diameter of 3.5 μm+/−0.5 μm. These are reacted with a maleimide-thiol cross linker, Sulfo-SIAB (sulfosuccinimidyl (4-iodoacetyl)aminobenzoate; Thermo Fisher). Following this, the contrast agents are incubated with an equimolar amount of thiol-bearing antibody for 2 hours at room temperature on a rocker followed by four rounds of centrifugal washing in saline to remove unbound antibody. In order to maximize cost effectiveness, formulations are created with different concentrations of maleimide-bearing lipid incorporated in the shell (e.g., 0.5%, 1%, 2% and 5%) as a method to modulate the number of antibodies on each microbubble. These formulations are tested during the following in vitro binding assay. Controls are formulated in the same way, but do not include antibodies nor Sulfo-SIAB cross linker.

Verify Cellular Binding Affinity for Targeted Contrast Agents.

Cell culture studies are performed.

Cell Culture.

Rat hepatic endothelial cells are used to assess specific microbubble adhesion. Cells are grown to confluency in high-glucose Dulbecco modified Eagle medium supplemented with 10% heat inactivated fetal bovine serum and 1% penicillin-streptomycin (Gibco, Grand Island, N.Y.), and maintained at 37° C. in a 95% air/5% CO₂ environment.

To assess non-specific contrast agent adhesion, rat extracellular matrix (ECM) is used. Decellularized livers are obtained and sliced into thin 50 μm sections using a vibrating microtome. These 50 μm matrix slabs are stored in the same conditions as the cells.

Binding Assay.

Plates containing either cells or ECM are exposed to one of the five microbubble solutions (controls, and 0.5-5% antibody coverage). Microbubbles are diluted in saline and allowed to incubate with the target (cells or ECM) for 3 minutes. During the incubation, the plates are inverted to allow contrasts agents to float upward making direct contact with the target. The plates are then placed in the upright position and gently washed with saline to remove any non-targeted contrast agents. The plates are then imaged with a microscope fitted with 60× immersion lens, and ten randomly selected fields of view per dish are digitally recorded. Each microbubble concentration (control, 0.5%, 1%, 2%, and 5% antibody coverage) is tested on four different samples of the two targets (cells or ECM) yielding a total of experiments (5 bubble types×2 target types×4 repetitions).

Data Analysis.

The binding affinity of each contrast agent formulation is quantified offline using a custom Matlab script by counting the number of contrast agents bound within the field of view (confluent cell growth enables us to compare bubble binding results between cell plates). This is an assay that has been previously performed, albeit for a different formulation of contrast agents. It is anticipated that increasing the percent of the microbubble shell with antibody coverage will correlate with more microbubbles bound per cell. The optimal antibody concentration is selected using a curve fit y(t)=A*(1−e^{-t/B}) to these data in order to determine the concentration at which 95% of cells are predicted to have at least one contrast agent bound to them (specific adhesion), but no more than 5% of the square area of ECM has contrast agent bound to it (non-specific adhesion).

Contrast agents produced with a pre-determined minimum concentration of bubbles/mL with diameter distribution of 3.5 μm+/−0.5 μm for maleimide lipid concentrations of 0% (control bubbles), 0.5%, 1%, 2%, and 5%. Bubble-target adhesion assay performed on 20 samples of cells and 20 samples of ECM. Curve fits to resulting data, and selection of antibody concentration which is predicted to result in 95% of endothelial cells having at least 1 bound bubble without exceeding threshold of 1 bound bubble per 5% of square area of ECM.

### Example 5

### Comparison of Noninvasive Imaging Performance Against Histology for Reseeding of Rat Liver

Pending successful results from the preceding EXAMPLE, the last step to demonstrating feasibility of the presently disclosed techniques is imaging recellularized scaffolds and comparing against the current gold standard: histology. Validation against histology is important because it is the primary method by which tissue engineers assess their organ constructs.

This EXAMPLE demonstrates that noninvasive endothelial cell imaging of recellularized rat liver scaffolds matches histologically determined endothelialization. This is accomplished by imaging six rat liver scaffolds that have been seeded and immediately sacrificing the organs for histology following imaging. Success is measured by how well noninvasive imaging matches histology.

Perform Longitudinal Ex Vivo Imaging Study During Recellularization Procedure.

Six livers from healthy Sprague-Dawley rats are acquired (Charles River) and prepared for a decellularization/recellularization procedure following known methods. After euthanizing an animal, the liver is surgically removed, and one side of vena cava is ligated while the other end along with the portal vein is fitted with 20-gauge cannulae and tubing. Decellularization occurs via perfusion with water and a mild detergent (Triton-X 100 with 0.1% Ammonium Hydroxide) over the course of 24 hours. Finally, livers are moved to a bioreactor of the presently disclosed subject matter and endothelial cell seeding is performed by injecting 30×10⁶ human umbilical vein endothelial cells (hUVECs) through the portal vein of the scaffold in addition to Advanced RPMI with 10% FBS, 1% antibiotics (Invitrogen, Corp., Carlsbad, Calif.), and a growth factor solution over a period of 16 hours with a peristaltic pump set to 3 mL/min. Once seeding is complete, the pump is set to 0.5 mL/min for constant perfusion for 5 days.

Imaging Protocol.

Prior to the onset of recellularization, each decellularized organ is imaged using conventional untargeted 3D acoustic angiography to capture baseline vascular images as per Gessner et al. (2013) 34 Biomaterials 9341-9351, the disclosure of which is incorporated herein in its entirety. Once recellularization has begun, one organ is imaged at random following the molecular imaging workflow described herein above each day of the 5-day protocol. Following imaging, the chosen organ is immediately sacrificed for histology and fixed in 4% paraformaldehyde. Additionally, one organ is sacrificed prior the onset of recellularization resulting in six timepoints with matched histology (i.e., baseline, day 1, day 2, etc.). Captured data sets are saved to the hard drive of a computer and analyzed offline.

To compute percent endothelialization, each molecular acoustic angiography image is compared against its untargeted baseline using the following methods. First, the TIFF stack from untargeted imaging is loaded into Matlab and thresholded to create binary image masks representing only pixels residing inside a vessel. Next, the perimeter of the vessel masks in each 2D slice are identified automatically using morphological erosion (with 4×4 kernel size). The same procedure is performed on the molecular angiography images (threshold, binary mask, detect perimeters) and the ratio of total perimeter from the molecular image to the untargeted image is considered the percent endothelialization metric from ultrasound.

Confirm Presence and Degree of Endothelialization Via Standard Histology protocols.

As mentioned above, time-matched histology is collected following each imaging session by sacrificing one whole organ scaffold. From each fixed sample, a 5 mm portion of each of the seven lobes of the liver is embedded in paraffin and sectioned for immunohistochemistry. Two slides are prepared from each block including H&E and CD31 antibody staining (total of 14 slides per organ). Slides are digitized, and an experienced pathologist blinded to the imaging results performs morphometric analysis to measure percent endothelialization using conventional methods.

Determine Correlation Between Noninvasive Imaging and Histology.

The agreement between ultrasound and histology is tested by performing linear regression between the two data sets. Data will be pooled such that mean percent endothelization across all seven liver lobes from histology is compared to overall percent endothelialization from ultrasound. Achieving good agreement (R²≥0.8) between ultrasound and histology measurements of percent endothelialization indicates that this aim has succeeded.

It is expected that the cell-seeding procedure endothelializes the liver vasculature uniformly and that histological sections represent the overall cell seeding efficacy for a given time point. If certain lobes of the liver are not seeded properly and/or R² between ultrasound and histology is below 0.8, the analysis of the ultrasound data is limited to regions specifically harvested for histology. If microbubble targeting is weak or non-existent, microbubble concentrations employed are increased 10-fold.

### Example 6

### Imaging of a Kidney Sample

FIG. 21 depicts another example of an imaging system of the presently disclosed subject matter as employed to image an explanted porcine kidney sample. The kidney sample (organ) was immobilized in a holder, with the holder connected to a robotic stage that moved the kidney sample relative to the ultrasound transducer in two dimensions (axes of robotic stages). FIG. 22 are images of the porcine kidney sample using an exemplary imaging system of the presently disclosed subject matter. The gray areas correspond to kidney tissue. The black arrows indicate areas of accumulation of contrast along vessel walls in the kidney sample (the corresponding regions appear yellow in the corresponding color images).

### Discussion of the Exemplar Embodiments

Summarily, in some embodiments the components of the systems and methods of the presently disclosed subject matter provide at least the following advantages over current imaging technologies:
1. hardware to allow internal contents of a bioreactor to be imaged via ultrasound or other visualization techniques without loss of sterility;
2. bioreactor hardware that incorporates acoustically transparent walls;
3. software to allow ultrasound or other acoustic data to map to different imaging modalities based on fiducial alignment points on the hardware, or using the 3D image data itself;
4. imaging acquisition and processing systems and methods to determine the extent of endothelial cell coverage within a tissue scaffold in real time;
5. hardware for preparation and injection of microbubble contrast in an automated way to ensure neither too much nor too little contrast enters the tissue scaffold;
6. methods to align 3D images of the organ to itself between timepoints in order to automatically map changes in ultrasound-derived metrics over time;
7. methods for allowing multiple bioreactors to dock to the imaging component of the presently disclosed systems;
8. methods for chemically inducing a marker on cell to promote adhesion of a contrast agent;
9. using radiation force to increase binding efficiency of contrast agents to walls;
10. real time targeted imaging using moving minimum filter to detect stationary contrast agents. Stationary contrast agents=stationary cells=identification of seeded endothelial cells;
11. contrast agents targeted to CD31 or other endothelial cell markers to visualize regions of endothelial cell coverage in 3D;
12. contrast agents targeted to extracellular matrix markers to visualize regions that lack endothelial cell coverage in 3D;
13. non-specific targeted contrast agents used to normalize for non-specific adhesion;
14. methods for quantifying vessel network patency; and
15. method for clearing contrast agents from the interior of the bioreactor to thereby enhance signal-to-noise ratios.

What is claimed is:

1. A system for analyzing cell distribution in an engineered tissue sample present within a bioreactor, the system comprising:
(a) an imaging system comprising at least one ultrasound transducer for acquiring ultrasound images from an engineered tissue sample present in the bioreactor; and
(b) a processing unit configured to analyze the ultrasound images acquired by the ultrasound transducer from the engineered tissue sample in order to output measured characteristics of the engineered tissue sample.

2. The system of claim 1 wherein the ultrasound transducer is configured be located external to the bioreactor when acquiring the ultrasound images.

3. The system of claim 2 wherein the ultrasound transducer is configured to obtain the ultrasound images through an acoustically transmissive window in the bioreactor.

4. The system of claim 1 wherein the ultrasound transducer is configured to be located in the bioreactor when acquiring the ultrasound images.

5. The system of claim 4 wherein the bioreactor comprises a three dimensional printer for generating the engineered tissue sample through a three dimensional printing process.

6. The system of claim 5 wherein the ultrasound transducer is interchangably couplable to the three dimensional printer for acquiring the ultrasound images.

7. The system of claim 5 wherein the ultrasound transducer is separate from the three dimensional printer for acquiring the ultrasound images.

8. The system of claim 1 wherein the ultrasound transducer is configured to generate ultrasound energy to image an ultrasound contrast agent configured to bind to the engineered tissue sample.

9. The system of claim 8 wherein the processing unit is configured to output an indication of an amount of the ultrasound contrast agent bound to the engineered tissue sample.

10. A system for analyzing cell distribution in an engineered tissue sample, the system comprising:
(a) a bioreactor for generating an engineered tissue sample, wherein the bioreactor:
(i) comprises an interior region for holding the engineered tissue sample;
(ii) comprises one or more input lines and one or more exit lines, both in fluid communication with the bioreactor for introducing a fluid into the interior region and removing the fluid from the interior region, and
(iii) comprises a window that is transmissive to ultrasound waves;
(b) a pump connected to at least one of the one or more input lines and/or to at least one of the one or more exit lines configured to regulate flow of the fluid into and out of the interior region; and
(c) an imaging system comprising at least one ultrasound transducer for acquiring ultrasound images from the engineered tissue sample present in the bioreactor.

11. The system of claim 10, further comprising a processing unit configured to analyze the ultrasound images acquired from the engineered tissue sample in order to output measured characteristics of the engineered tissue sample.

12. The system of claim 11, wherein at least one of the one or more input lines comprises an inlet port configured to permit introduction of a reagent into the fluid under conditions such that the reagent perfuses the engineered tissue sample.

13. The system of claim 12, wherein the reagent comprises a contrast agent.

14. The system of claim 13, wherein the contrast agent comprises a ligand that specifically binds to a target molecule present in the engineered tissue sample.

15. The system of claim 14, wherein the ligand comprises an antibody or an antigen-binding fragment thereof that specifically binds to the target molecule.

16. The system of claim 14, wherein the target molecule is present in the engineered tissue sample and is accessible to the ligand in locations of the engineered tissue sample that are decellularized or non-cellularized.

17. The system of claim 16, wherein the ligand binds to a collagen matrix present in a decellularized or non-cellularized region of the engineered tissue sample.

18. The system of claim 14, wherein the target molecule is present in the engineered tissue sample and is accessible to the ligand in locations of the engineered tissue sample that are recellularized.

19. The system of claim 18, wherein the target molecule is present in the engineered tissue sample only in locations of the engineered tissue sample that are recellularized.

20. The system of claim 14, wherein the target molecule is a molecule expressed by an endothelial cell.

21. The system of claim 20, wherein the molecule expressed by an endothelial cell is selected from the group consisting of CD31, P-selectin, E-selectin, VEGF-R2, and αᵥβ₃ integrin.

22. The system of claim 10, wherein the flow of the fluid in the bioreactor is interruptible to stop perfusion of the engineered tissue sample.

23. The system of any one of the preceding claims, wherein the engineered tissue sample comprises a liver scaffold, a lung scaffold, or a kidney scaffold.

24. The system of claim 23, wherein the engineered tissue sample is decellularized.

25. The system of any one of the preceding claims, wherein the engineered tissue sample comprises a bioprinted organ or tissue.

26. The system of claim 10, wherein the fluid carries an ultrasound contrast agent through the engineered tissue sample, and wherein at least one of the at least one exit lines is configured to selectively route output of fluid from the bioreactor to remove a portion of the contrast agent that does not bind with the engineered tissue sample from the bioreactor.

27. The system of any one of the preceding claims, wherein the ultrasound transducer is connected to the bioreactor via a docking mechanism that permits two-dimensional or three-dimensional movement of the ultrasound transducer relative to the engineered tissue sample.

28. The system of any one of the preceding claims, wherein the ultrasound transducer is capable of receiving ultrasound signals of >5 MHz.

29. The system of any one of the preceding claims, wherein the processing unit is configured to accept ultrasound image input and output percent cellularization of the engineered tissue sample.

30. A method for analyzing distribution of cells in an engineered tissue sample present within a bioreactor, the method comprising:
(a) introducing a contrast agent to perfusion input of the engineered tissue sample, wherein the contrast agent specifically binds to a target molecule expressed by endothelial cells present within the engineered tissue sample or to a target molecule present in a decellularized region of the engineered tissue sample;
(b) permitting the contrast agent to contact the engineered tissue sample under conditions and for a time sufficient to allow binding of the contrast agent to the target molecule, if present; and
(c) acquiring image data of the engineered tissue sample,
wherein the image data allows for a determination of whether or not the contrast agent has bound to the engineered tissue sample in one or more regions of the engineered tissue sample.

31. The method of claim 30, further comprising processing the acquired image data using a processing unit capable of transforming the acquired image data into output indicative of one or more regions of the engineered tissue sample where endothelial cells are or are not present.

32. The method of claim 31, wherein the acquired image data is outputted as spatial density of endothelial cells based on the image of stationary contrast agents, optionally in comparison to reference image data.

33. The method of claim 31, wherein the acquired image data is outputted as spatial density of decellularized regions of the engineered tissue sample, thereby providing a map of a network of decellularized vasculature of the engineered tissue sample.

34. The method of any one of claims 30-34, wherein the engineered tissue sample comprises a bioprinted organ or tissue sample.

35. A method for analyzing a bioprinted organ or tissue sample, the method comprising:
(a) introducing a contrast agent to perfusion input of the bioprinted organ or tissue sample, wherein the contrast agent specifically binds to a target molecule expressed by cells present within the bioprinted organ or tissue sample;
(b) permitting the contrast agent to contact the bioprinted organ or tissue sample under conditions and for a time sufficient to allow binding of the contrast agent to the target molecule, if present; and
(c) acquiring image data of the bioprinted organ or tissue sample,
wherein the image data allows for a determination of whether or not the contrast agent has bound to the bioprinted organ or tissue sample in one or more regions of the bioprinted organ or tissue sample.

36. The method of claim 35, further comprising processing the acquired image data using a central processing unit programmed with software capable of transforming the acquired image data into output of one or more regions of the bioprinted organ or tissue sample where cells are or are not present.

37. The method of claim 36, wherein the acquired image data is outputted as spatial density of cells based on the image of stationary contrast agents, optionally in comparison to reference image data.

38. The method of claim 36, wherein the acquired image data is outputted as spatial density of non-cellularized and/or incompletely cellularized regions of the bioprinted organ or tissue sample, thereby providing a map of a network of non-cellularized and/or incompletely cellularized regions of the bioprinted organ or tissue sample.

39. The method of claim 36, wherein acquiring the image data includes using an ultrasound transducer located external to a bioreactor in which the bioprinted organ or tissue sample is located.

40. The method of claim 36, wherein acquiring the image date includes using an ultrasound transducer located inside of a bioreactor in which the bioprinted organ or tissue sample is located.
